(19) United States
(12) Patent Application Publication
(10) Pub. No.: US 2017/0119841 A1
(43) Pub. Date: May 4, 2017

(54) AMINO ACID SALTS OF UNSATURATED FATTY ACIDS

(71) Applicant: Thetis Pharmaceuticals LLC, Southport, CT (US)

(72) Inventors: Gary Mathias, Ridgefield, CT (US); Frank C. Sciavolino, Waterford, CT (US)

(21) Appl. No.: 15/342,420

(22) Filed: Nov. 3, 2016

Related U.S. Application Data
(60) Provisional application No. 62/250,654, filed on Nov. 4, 2015.

(51) Int. Cl. A61K 38/05; A61K 45/06; C07K 5/078; C07K 5/062; C07K 5/06

(52) U.S. Cl. CPC A61K 38/05; C07K 5/06026; C07K 5/06052; C07K 5/06043; C07K 5/06008; C07K 5/06147; A61K 45/06

(57) ABSTRACT

The present invention provides compounds of Formula I and Formula II and related compositions and methods.

### FIELD OF THE INVENTION

The present invention relates to compounds which are amino acid and dipeptide salts of fatty acids, compositions comprising same, and methods for their manufacture and use.

### BACKGROUND

Polyunsaturated fatty acids of the omega-3 series (“omega-3 fatty acids”) have shown a wide spectrum of biological activities suggesting their possible usefulness in treating a range of diseases and disorders including metabolic disorders, cardiovascular complications, inflammatory diseases, central nervous system disorders, and ophthalmic complications. There are three major types of omega-3 fatty acids involved in the human physiology: α-linolenic acid (ALA; found in plant oils), eicosapentaenoic acid (EPA), and docosahexaenoic acid (DHA) (both commonly found in marine oils). Marine algae and phytoplankton are also sources of omega-3 fatty acids. Common sources of plant oils containing the omega-3 ALA fatty acid include walnut, edible seeds, clary sage seed oil, algal oil, flaxseed oil, Sacha Inchi oil, Echium oil, and hemp oil, while sources of animal omega-3 EPA and DHA fatty acids include fish oils, egg oil, squid oils, and krill oil. Often these primary omega-3 fatty acids are present with numerous minor omega-3 fatty acids as mixtures. But the poor aqueous solubility of omega-3 fatty acids limits their utility as therapeutic agents and as nutraceutical additives to food and drink due to a phenomenon referred to as solubility-limited absorption which limits the plasma levels that can be achieved following oral administration. In fact, the omega-3 fatty acids are essentially insoluble in water and both the free acid and sodium salt forms create soap-like emulsions when mixed with water. Thus, although omega-3 fatty acids are absorbed following oral administration, the relatively low plasma levels achieved cannot be increased simply by increasing the dose administered.

In addition to their poor aqueous solubility, omega-3 fatty acids suffer from susceptibility to lipid oxidation. This oxidation leads to formation of undesirable fishy and rancid off-flavors that render compositions comprising them less palatable.

WO 2014011895 describes fatty acid salts of eicosapentaenoic acid (EPA) with lysine or docosahexaenoic acid (DHA) or EPA with metformin, piperazine, and meglumine.

US 2011237813 (Jost Chemical Co.) describes mineral co-salts of polyunsaturated fatty acids and a non-fatty acid co-anion formed as a precipitate.

WO 2004082402 (Novartis AG) describes a combination, such as a combined preparation or pharmaceutical or nutritional composition, respectively, which comprises at least one cis-polyunsaturated fatty acid, at least one amino acid, and optionally at least one diabetes medicine for simultaneous, separate or sequential use in the prevention, delay of progression or treatment of diseases, especially metabolic disorders and in particular type 2 diabetes.

US 20140044828 describes nutritional compositions containing specified amounts of n-3 fatty acids and one or more of free lysine, dipeptides containing lysine, and lysine salts, for the prophylaxis and/or treatment of various symptoms associated with muscle mass decrease, decreased basal metabolism, and low body temperature, as well as for suppression of obesity, suppression of visceral fat accumulation, and treatment of hyperglycemia and hyperlipidemia, particularly in the elderly. The term “dipeptides containing lysine” is defined as referring to a dipeptide where at least one molecule of the dipeptide is lysine or a salt of lysine such as L-lysine hydrochloride, L-lysine acetate, and L-lysine glutamate. Lysyllysine is given as a specific example of a dipeptide containing lysine.

Lysyllysine is described in US 20080248564 in a process for the covalent modification of nucleic acids by lactosylation for more efficient transfer of nucleic acids into cells. This is described as an improvement over prior art methods relying on the formation of non-specific ionic complexes between nucleic acids and polycations such as polylysine, as described in U.S. Pat. No. 5,166,320. US 20060084617 describes the use of lysyllysine in a process for conjugating endosomolytic spermine to nucleic acids to enhance their delivery into cells.

US 20070275019 describes the preparation of vaccines directed to cancer-associated carbohydrate antigens, the vaccines comprising multivalent antigen systems in which lysyllysine is used as a core matrix bearing multiple antigens as dendritic arms.

There is a need to develop new compositions able to deliver fatty acids at much higher plasma levels than is possible using the currently available free fatty acid, sodium salt, or ester forms, in order to fulfill the therapeutic and nutritional promise of these compounds and translate the many promising in vitro and cellular pharmacology observations into clinical and general health benefits. The present invention addresses these needs.

### SUMMARY OF THE INVENTION

The disclosure provides compounds of Formula I and II, including enantiomers, polymorphs, solvates, and hydrates thereof, and mixtures thereof, as well as methods of making the compounds, compositions comprising same, and methods for their use. The compounds are chemically and physically stable solid materials, e.g., powders. In embodiments, the compounds also have low hygroscopicity and are highly water soluble. The compounds described here offer improved means for delivering fatty acids to a subject.

Compounds of Formula I have the structure:

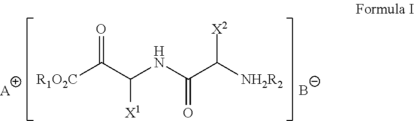

wherein
R₁ is H when A is absent, or absent when A is present;
R₂ is H or absent,
X₁ and X₂ may be the same or different, and are each the side chain of an amino acid residue,
A+ is present or absent and is a monovalent metal cation, or a non-metal molecule having at least one basic functionality, and
B− is a fatty acid molecule.

A compound of Formula I consists of at least (i) a dipeptide component and (ii) a fatty acid component (B−), with an optional basic moiety (A+). The dipeptide component contains X₁ and X₂ which may be the same or different, and are each the side chain of an amino acid residue. In embodiments, at least one of X₁ and X₂ is the side chain of an amino acid residue selected from serine, threonine, glycine, alanine, valine, leucine, isoleucine, methionine, and phenylalanine. In embodiments, where one of X₁ and X₂ is the side chain of an amino acid residue selected from serine, threonine, glycine, alanine, valine, leucine, isoleucine, methionine, and phenylalanine, the remainder of X₁ or X₂ is the side chain of an amino acid independently selected from lysine, arginine, histidine, aspartate, glutamate, serine, threonine, asparagine, glutamine, cysteine, glycine, proline, alanine, valine, isoleucine, leucine, methionine, phenylalanine, tyrosine, and tryptophan. In embodiments, the remainder is the side chain of lysine. In embodiments, at least one of X₁ and X₂ is the side chain of glycine, valine, serine, leucine, or histidine, and the remainder is the side chain of lysine.

A compound of Formula I may also optionally contain a molecule (A⁺) having at least one basic function which forms an ionic bond with the terminal carboxy of the amino acid component. In embodiments, A⁺ is a monovalent metal cation, e.g., Na⁺, K⁺, or a molecule having at least one basic functionality, such as a monovalent amine-based cation, e.g., tri-ethanolamine, or tri-ethylamine, or a basic pharmaceutical compound such as metformin or gabapentin.

As described in more detail below, the compounds of Formula I encompass simple salts of dipeptides and a fatty acid (Formula IA), simple metal salts of the dipeptides and a fatty acid with a monovalent metal (Formula IB), and simple non-metal salts of the dipeptides and a fatty acid with a non-metal molecule having at least one basic functionality (Formula IC).

Compounds of Formula II have the structure

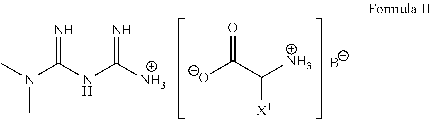

wherein

X₁ is the side chain of an amino acid residue,

B− is a fatty acid.

Compounds of Formula II share some basic features with compounds of Formula I, except that compounds of Formula II contain a single amino acid moiety and A+ is metformin.

The fatty acid component of the compounds described here may be selected from a naturally occurring, non-naturally occurring, branched or unbranched mono-, di- or poly-unsaturated fatty acid having from about 8 carbon atoms to about 24 carbon atoms. In embodiments, the fatty acid is selected from an omega-3 fatty acid, an omega-6 fatty acid, an omega-7 fatty acid, and an omega-9 fatty acid. In embodiments, the fatty acid is selected from eicosapentaenoic acid (EPA), docosahexaenoic acid (DHA), docosapentaenoic acid (DPA), hexadecatrienoic acid (HTA), α-Linolenic acid (ALA), stearidonic acid (SDA), eicosenoic acid, eicosatrienoic acid (ETE), all-cis-5,8,11-eicosatrienoic acid, eicosatetraenoic acid (ETA), heneicosapentaenoic acid (HPA), tetracosapentaenoic acid, tetracosahexaenoic acid, linoleic acid, gamma-linolenic acid (GLA), calendic acid, eicosadienoic acid, dihomo-gamma-linolenic acid (DGLA), arachidonic acid, adrenic acid, docosadienoic acid, docosapentaenoic acid (Osbond acid), tetracosapentaenoic acid, 24:5 (n-6), tetracosatetraenoic acid, palmitoleic acid, vaccenic acid, paullinic acid, oleic acid, elaidic acid, gondoic acid, mead acid, erucic acid, and nervonic acid.

In embodiments, the fatty acid component is a polyunsaturated fatty acid, particularly an omega-3 fatty acid selected from the group consisting of eicosapentaenoic acid (EPA), docosahexaenoic acid (DHA), docosapentaenoic acid (DPA), hexadecatrienoic acid (HTA), α-linolenic acid (ALA), stearidonic acid (SDA), eicosatrienoic acid (ETE), eicosatetraenoic acid (ETA), heneicosapentaenoic acid (HPA), tetracosapentaenoic acid, and tetracosahexaenoic acid. In a particular embodiment, the omega-3 fatty acid is selected from eicosapentaenoic acid (EPA), docosahexaenoic acid (DHA), and docosapentaenoic acid (DPA).

In embodiments, the fatty acid component is a polyunsaturated fatty acid, particularly an omega-6 fatty acid selected from the group consisting of linoleic acid, gamma-linolenic acid (GLA), eicosadienoic acid, dihomo-gamma-linolenic acid (DGLA), arachidonic acid, docosadienoic acid, adrenic acid, docosapentaenoic acid (Osbond acid), tetracosatetraenoic acid, and tetracosapentaenoic acid, 24:5 (n-6).

In embodiments, the fatty acid component is a polyunsaturated fatty acid, particularly an omega-9 fatty acid selected from the group consisting of mead acid, 20:3 (n-9), all-cis-5,8,11-eicosatrienoic acid, oleic acid, eicosenoic acid, erucic acid, and nervonic acid.

The disclosure also provides pharmaceutical compositions and dosage forms comprising or consisting of any of the compounds described herein, and mixtures thereof. In one embodiment, the dosage form is a solid dosage form. In embodiments, the solid dosage form is a powder, a tablet, a capsule, or a caplet. In embodiments, the solid dosage form, for example a powder, may be suitable for reconstitution in an aqueous vehicle, for example to be administered intraperitoneally.

In embodiments, the pharmaceutical composition or dosage form further comprises one or more optional excipients as described infra. In embodiments, the pharmaceutical composition or dosage form further comprises one or more additional active pharmaceutical agents (APIs). In embodiments, the one or more additional APIs is selected from the group consisting of an anti-hyperlipidemic agent, an anti-diabetic agent, an anti-epileptic agent, and an anti-inflammatory agent, and combinations thereof. In embodiments, the one or more additional APIs is an NSAID. In embodiments, the one or more additional APIs is an anti-hyperlipidemic agent selected from an HMG CoA enzyme inhibitor, a cholesterol absorption inhibitor, and a cholesterol esterase transfer protein (CETP) inhibitor, and combinations thereof. In one embodiment, the anti-hyperlipidemic agent is a statin. In embodiments, the statin is selected from the group consisting of atorvastatin, risuvostatin, simvastatin, pravastatin, and pharmaceutically acceptable salts or prodrugs thereof.

The disclosure also provides a food additive or dietary supplement comprising a compound of Formula I or Formula II, and mixtures thereof, optionally comprising a carrier suitable for administration to a human or non-human animal. In embodiments, the food additive or dietary supplement further comprises one or more additional biologically active agents. In embodiments, the one or more additional biologically active agents is selected from the group consisting of a vitamin, a mineral, an amino acid, a carbohydrate, an antioxidant, a flavonoid, a carotenoid, a phytoseterol, an herb, an enzyme, a botanical extract or concentrate, and a botanical compound, and combinations thereof. In embodiments, the one or more additional biologically active agents is selected from the group consisting of vitamin A, vitamin B1, vitamin B12, vitamin B6, vitamin C, vitamin D, vitamin E, vitamin K, calcium, carnitine, chromium, chondroitin, coenzyme Q10 (ubiquinone), folate, glucosamine, metafolin, riboflavin, biotin, iodine, iron, magnesium, selenium, thiamin, and zinc, and combinations thereof.

The disclosure also provides unit dosage forms comprising the compounds or mixtures of the compounds described here. In embodiments, the unit dosage form comprises from about 0.05 g to 12 g of fatty acids total as the fatty acid component of the compound or mixture of compounds in the unit dosage form.

The disclosure also provides pharmaceutical and non-pharmaceutical uses of the compounds and mixtures of compounds described herein. In embodiments, a compound or composition comprising same is useful for delivering the fatty acids of the fatty acid component as free fatty acids, or mixtures of two or more different free fatty acids, in ionic form to a subject. In embodiments, a compound or composition of the invention is also useful for delivering, along with the free fatty acids, or mixture of free fatty acids in ionic form, at least one additional API or biologically active agent to a subject. In embodiments, the free fatty acids delivered are medium or long chain fatty acids as described above and infra. In embodiments, the at least one additional API or biologically active agent is as described above and infra.

In embodiments, a compound or composition of the disclosure is useful for treating a disease or disorder responsive to treatment with a polyunsaturated fatty acid. In embodiments, at least 50 wt %, at least 60 wt %, at least 70 wt %, or at least 90 wt % of the fatty acid component of the compound or composition consists of one or more omega-3 fatty acids independently selected from eicosapentaenoic acid (EPA), docosahexaenoic acid (DHA), and docosapentaenoic acid (DPA). In embodiments, the disease or disorder is selected from a gastrointestinal inflammatory disease or disorder, a metabolic disease or disorder, a cardiovascular disease or disorder, a hematological disorder, cancer, an inflammatory disease or disorder, and a neurological disease or disorder.

In embodiments, the gastrointestinal inflammatory disease or disorder is postoperative intestinal inflammation, postoperative ileus, ischemia reperfusion injury, or a combination thereof. In one embodiment, the gastrointestinal inflammatory disease or disorder is post-operative ileus (POI).

In embodiments, the metabolic disease or disorder is abnormal glucose metabolism manifesting in diabetes, including type 2 diabetes, or pre-diabetes, insulin resistance, abnormal lipid metabolism manifesting as hypertriglyceridemia, i.e., elevated triglycerides, mixed dyslipidemia, hypercholesterolemia, fatty liver, and combined abnormal glucose and lipid metabolism manifesting in obesity; or a dyslipidemic disorder selected from hypertriglyceridemia, hypercholesterolemia and mixed dyslipidemias.

In embodiments, the metabolic disease or disorder is hypertriglyceridemia, severe hypertriglyceridemia, hypercholesterolemia, pre-diabetes, fatty liver disease, or obesity.

In embodiments, the cardiovascular disease or disorder is atrial fibrillation, myocardial infarction, or congestive heart failure.

In embodiments, the hematological disorder is sickle cell disease.

In embodiments, the inflammatory disease or disorder is arthritis, inflammatory bowel disease, or psoriasis.

In embodiments, the inflammatory disease or disorder is an ophthalmic inflammation disorder or dry eye syndrome.

In embodiments, the neurological disease or disorder is a psychiatric disorder selected from Alzheimer's disease, attention deficit hyperactivity disorder (ADHD) or depression. In one embodiment, the neurological disease or disorder is a neuro trauma injury selected from traumatic brain injury, spinal cord injury, ischemic stroke, or concussion.

In embodiments, the disclosure provides a method for treating nociceptive pain, the method comprising administering to a subject in need of such treatment, a compound of Formula I, or a composition comprising same. In embodiments, the fatty acid component consists of an omega-3 fatty acid, preferably selected from eicosapentaenoic acid (EPA), docosahexaenoic acid (DHA), and docosapentaenoic acid (DPA), and mixtures thereof. In embodiments, the composition further comprises gabapentin.

In embodiments, the disclosure provides a method for treating neuropathic pain, the method comprising administering to a subject in need of such treatment, a compound of Formula I, or a composition comprising same. In embodiments, the fatty acid component consists of an omega-3 fatty acid, preferably selected from eicosapentaenoic acid (EPA), docosahexaenoic acid (DHA), and docosapentaenoic acid (DPA), and mixtures thereof. In embodiments, the composition further comprises an NSAID.

In embodiments, the disclosure provides a method for treating epilepsy or epileptic syndrome, the method comprising administering to a subject in need of such treatment, a compound of Formula I, or a composition comprising same. In embodiments, the fatty acid component consists of an omega-3 fatty acid, preferably selected from eicosapentaenoic acid (EPA), docosahexaenoic acid (DHA), and mixtures thereof. In embodiments, the composition further comprises gabapentin.

The disclosure also provides for the use of a compound of Formula I as a food additive or dietary supplement. In embodiments, the use is to counter a dietary deficiency or nutritional disorder in a subject, or in a method for maintaining, promoting, or improving the general health of a subject. Accordingly, the invention provides methods of countering a dietary deficiency or nutritional disorder in a subject, as well as methods for maintaining, promoting, or improving the general health of a subject, the methods comprising administering to the subject a compound of Formula I, or a composition or unit dosage form comprising same, or a composition comprising a mixture of two or more of said compounds. In embodiments, the composition comprises from 50 mg to 6 g of EPA, DHA, DPA, or total fatty acids, preferably total polyunsaturated fatty acids, as the fatty acid component of the compounds in the composition. In embodiments, the fatty acids are omega-3, omega-6, omega-7, or omega-9 series fatty acids, or mixtures of two or more of the foregoing. In embodiments, the fatty acids comprise or consist of one or more omega-3 fatty acids independently selected from eicosapentaenoic acid (EPA), docosahexaenoic acid (DHA), and docosapentaenoic acid (DPA). In embodiments, at least 50% of the fatty acid component of the compounds in the composition consists of one or more omega-3 fatty acids independently selected from eicosapentaenoic acid (EPA), docosahexaenoic acid (DHA), and docosapentaenoic acid (DPA).

In one embodiment, the method is a method for improving prenatal health. In one embodiment of this method, the composition comprises from 50 mg to 6 g of DHA or total omega-3 fatty acids as the fatty acid component of the compounds in the composition, and optionally further comprises one or more of a B vitamin, vitamin C, vitamin E, vitamin A, vitamin D, iron, zinc, calcium, iodine, metafolin, methylsulfonylmethane (also known as dimethyl sulfone and methyl sulfone), N-acetyl-L-cysteine, green tea extract (Camellia sinensis), and grape seed extract (Vitis vinifera). In one embodiment, the B vitamin is selected from thiamine (vitamin B-1), riboflavin (vitamin B-2), niacin (vitamin B-3), pantothenic acid (vitamin B-5), biotin (vitamin B-7), and folic acid (vitamin B-9), or any combination of the foregoing.

In one embodiment, the method is a method for improving heart health. In one embodiment of this method, the composition comprises from 50 mg to 6 g of EPA or total omega-3 fatty acids as the fatty acid component of the compounds in the composition, and optionally further comprises one or more of coenzyme Q10, L-carnitine, an antioxidant, a phytoseterol, and a flavonoid.

In one embodiment, the method is a method for improving joint health. In one embodiment of this method, the composition comprises from 50 mg to 6 g of EPA or total omega-3 fatty acids as the fatty acid component of the compounds in the composition, and optionally further comprises one or more of chondroitin, glucosamine sulfate, calcium, vitamin D3, ginger extract, turmeric, curcumin, collagen, and a non-steroidal anti-inflammatory (NSAID).

In one embodiment, the method is a method for improving eye health. In one embodiment of this method, the composition comprises from 50 mg to 6 g of DHA or total omega-3 fatty acids as the fatty acid component of the compounds in the composition, and optionally further comprises one or more of vitamin A, vitamin C, vitamin E, calcium, zinc, copper, selenium, a carotenoid, a flavonoid, and folic acid.

In one embodiment, the method is a method for improving cognitive health. In one embodiment of this method, the composition comprises from 50 mg to 6 g of EPA or total omega-3 fatty acids as the fatty acid component of the compounds in the composition.

### DETAILED DESCRIPTION OF THE INVENTION

The disclosure provides compounds of Formula I and II and related compositions as well as methods for making the compounds and methods for their use. As described in more detail in the following sections, the disclosure provides compositions comprising one or more of the compounds described herein, and mixtures thereof. Also provided are compositions further comprising a second active agent. In embodiments the second active agent is a biologically active agent or an active pharmaceutical ingredient (API), as described herein.

In embodiments, the composition is a pharmaceutical composition. In other embodiments, the composition is a dietary additive or supplement. In embodiments, mixtures of two or more of the compounds described here are prepared by physical admixture of the compounds in the desired proportions.

The disclosure also provides a package or kit comprising a unit dosage form of a compound or composition described herein, at least one container for holding the unit dosage form, and instructions for use.

The compounds of Formula I and II each contain at least (i) an amino acid component, and (ii) a fatty acid component (B−). The compounds may also optionally contain a molecule (A+) having at least one basic function. In embodiments A+ is a monovalent metal cation, or a non-metal molecule having at least one basic function. In embodiments the non-metal compound is a primary amine, a secondary amine, a tertiary amine, or a guanidine. In one embodiment, the non-metal compound is tri-ethanolamine. The non-metal compound may also be a therapeutic agent having a basic functionality, for example metformin or gabapentin.

The compounds described here advantageously provide the fatty acid component in the form of a chemically and physically stable solid material, such as a powder. The fatty acid component of the compounds described herein is both water soluble and stable against oxidative degradation. These compounds thus provide considerable advantages over fatty acid compositions which are in the physical form of an oily liquid which is relatively difficult to formulate and chemically susceptible to degradation, especially oxidative degradation. The physical properties of the compounds of the present disclosure are described in more detail infra.

### Compounds of Formula I

In embodiments, the invention provides a compound of Formula I, and enantiomers, polymorphs, solvates, and hydrates thereof:

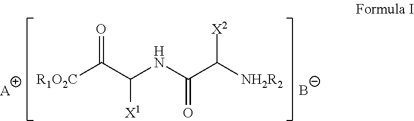

wherein
R₁ is H when A is absent, or absent when A is present,
R₂ is H or absent,
X₁ and X₂ may be the same or different and are each the side chain of an amino acid residue,
A+ is present or absent and is a monovalent metal cation, or a non-metal molecule having at least one basic function, and
B− is a fatty acid molecule.

A compound of Formula I consists of at least (i) a dipeptide component and (ii) a fatty acid component (B−), with an optional basic moiety (A+). The dipeptide component contains X₁ and X₂ which may be the same or different, and are each the side chain of an amino acid residue. In embodiments, at least one of X₁ and X₂ is the side chain of an amino acid residue selected from serine, threonine, glycine, alanine, valine, leucine, isoleucine, methionine, and phenylalanine. In embodiments, where one of X₁ and X₂ the side chain of an amino acid residue selected from serine, threonine, glycine, alanine, valine, leucine, isoleucine, methionine, and phenylalanine, the remainder of X₁ or X₂ is the side chain of an amino acid residue independently selected from lysine, arginine, histidine, aspartate, glutamate, serine, threonine, asparagine, glutamine, cysteine, glycine, proline, alanine, valine, isoleucine, leucine, methionine, phenylalanine, tyrosine, and tryptophan. In embodiments, the remainder is the side chain of lysine. In embodiments, at least one of X₁ and X₂ is the side chain of glycine, valine, serine, leucine, or histidine, and the remainder is the side chain of lysine.

A compound of Formula I may also optionally contain a molecule (A⁺) having at least one basic function. In embodiments, A⁺ is a monovalent metal cation, e.g., Na⁺, K⁺, or a molecule having at least one basic functionality, such as a monovalent amine-based cation, e.g., tri-ethanolamine, or tri-ethylamine or a basic pharmaceutical compound such as metformin or gabapentin.

As described in more detail below, the compounds of Formula I encompass simple salts of dipeptides and a fatty acid (Formula IA), simple monovalent metal salts of the dipeptides and a fatty acid (Formula IB), and simple non-metal salts of the dipeptides and a fatty acid with a non-metal molecule having at least one basic functionality (Formula IC).

The following non-limiting examples of compounds of Formula I is provided to illustrate the nature of the compounds described and is not intended to limit the disclosure to the particular compounds depicted.

A compound of Formula I wherein

R₁ and R₂ are each H,

X₁ and X₂ are each H,

A+ is absent,

B− is a fatty acid,

and enantiomers, polymorphs, solvates, and hydrates thereof:

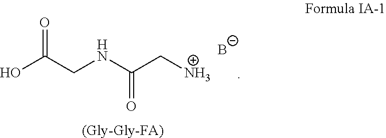

A compound of Formula I wherein

R₁ and R₂ are each H,

X₁ is the side chain of lysine (butylamine),

X₂ is the side chain of valine (isopropyl),

A+ is absent,

B− is a fatty acid,

and enantiomers, polymorphs, solvates, and hydrates thereof:

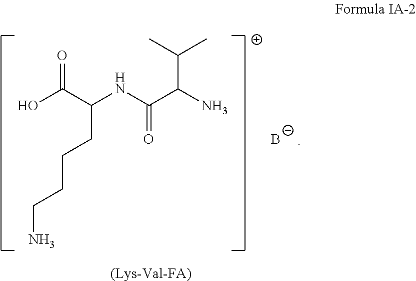

A compound of Formula I wherein

R₁ and R₂ are each H,

X₁ is the side chain of lysine (butylamine),

X₂ is the side chain of serine,

A+ is absent,

B− is a fatty acid,

and enantiomers, polymorphs, solvates, and hydrates thereof:

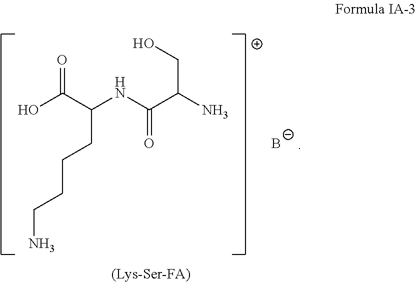

A compound of Formula I wherein

R₁ and R₂ are each H,

X₁ is the side chain of lysine (butylamine),

X₂ is the side chain of glycine

A+ is absent,

B− is a fatty acid,

and enantiomers, polymorphs, solvates, and hydrates thereof:

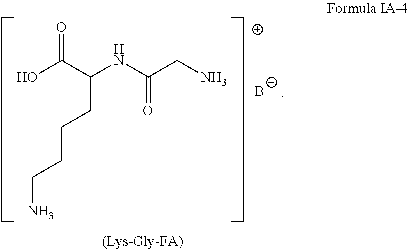

A compound of Formula I wherein

R₁ and R₂ are each H,

X₁ is the side chain of lysine (butylamine),

X₂ is the side chain of leucine (isobutyl)

A+ is absent,

B− is a fatty acid,

and enantiomers, polymorphs, solvates, and hydrates thereof:

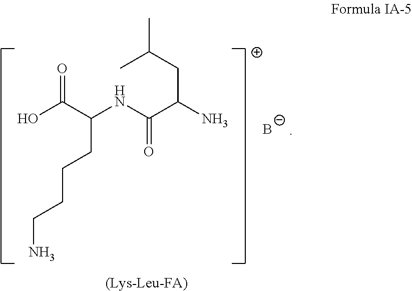

A compound of Formula I wherein

R₁ and R₂ are each H,

X₁ is the side chain of lysine (butylamine),

X₂ is the side chain of histidine (imidazole)

A+ is absent,

B− is a fatty acid,

and enantiomers, polymorphs, solvates, and hydrates thereof:

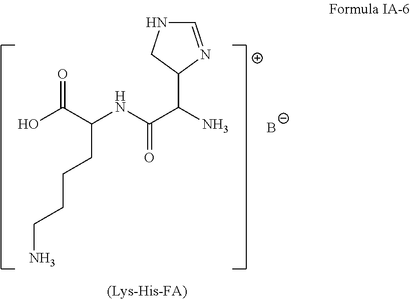

In embodiments, A+ is a monovalent metal cation such as Na⁺ or K⁺ (Formula IB). Accordingly, the disclosure provides compounds of Formula IB including the following:

A compound of Formula I wherein

R₁ is absent,

R₂ is H,

X₁ and X₂ are each H,

A+ is Na⁺,

B− is a fatty acid

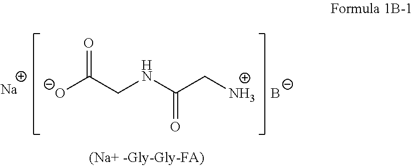

In embodiments, A+ is a non-metal molecule having at least one basic functionality, such as a monovalent amine-based cation, e.g., tri-ethanolamine, or tri-ethylamine or a basic pharmaceutical compound such as metformin or gabapentin. Accordingly, the disclosure provides compounds of Formula IC including the following:

A compound of Formula I wherein

R₁ is absent,

R₂ is H,

X₁ and X₂ are each H,

A+ is a non-metal and a molecule having at least one basic functionality,

B− is a fatty acid,

and enantiomers, polymorphs, solvates, and hydrates thereof:

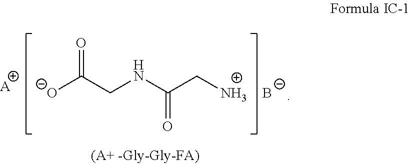

A compound of Formula I wherein

R₁ is absent,

R₂ is H,

X₁ and X₂ are each H,

A+ is trienthanolamine,

B− is a fatty acid,

and enantiomers, polymorphs, solvates, and hydrates thereof:

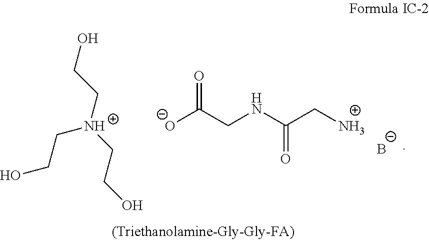

A compound of Formula I wherein

R₁ is absent,

R₂ is H,

X₁ and X₂ are each H,

A+ is metformin,

B− is a fatty acid,

and enantiomers, polymorphs, solvates, and hydrates thereof:

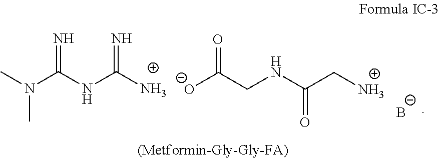

### Compounds of Formula II

In embodiments, the invention provides a simple amino acid salt of metformin with a fatty acid (Formula II), and enantiomers, polymorphs, solvates, and hydrates thereof. A compound of Formula II has the following structure:

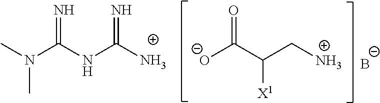

wherein

X₁ is the side chain of an amino acid residue,

B− is a fatty acid.

### The Fatty Acid Component of Formula I and Formula II Compounds

The term “fatty acid” is used to describe a carboxylic acid with a long aliphatic carbon chain of from about 4 to 28 carbon atoms, which is either saturated or unsaturated, referring to whether the carbon chain contains one or more double bonds between the carbon atoms (unsaturated). In one embodiment, the fatty acid is an unsaturated fatty acid. In one embodiment, the unsaturated fatty acid is a mono-, di-, or polyunsaturated fatty acid. In one embodiment, the fatty acid is a polyunsaturated fatty acid. In one embodiment, the polyunsaturated fatty acid is a long-chain polyunsaturated fatty acid having 16 to 24 carbon atoms (C₁₆-C₂₄), or 20 to 22 carbon atoms (C₂₀-C₂₂). In one embodiment, the polyunsaturated fatty acid is a medium-chain polyunsaturated fatty acid having 8 to 15 carbon atoms (C₈-C₁₅), or 8 to 12 carbon atoms (C₈-C₁₂).

In one embodiment, the polyunsaturated fatty acid is a fatty acid of the omega-3, omega-6, omega-7, or omega-9 series. In one embodiment, the fatty acid is selected from a mono-, di-, or polyunsaturated fatty acid of the omega-3, omega-6, omega-7, or omega-9 series. Examples of fatty acids of the omega-3, 6, 7, and 9 series are provided in Table 1, below. In one embodiment, the fatty acid is selected from a fatty acid set forth in Table 1.

TABLE 1
Fatty acids (mono- and di-unsaturated) of the omega-3, 6, 7, and 9 series.

| Common name | Lipid name | Chemical name |
|---|---|---|
| Hexadecatrienoic acid (HTA) | 16:3 (n-3) | all-cis-7,10,13-hexadecatrienoic acid |
| α-Linolenic acid (ALA) | 18:3 (n-3) | all-cis-9,12,15-octadecatrienoic acid |
| Stearidonic acid (SDA) | 18:4 (n-3) | all-cis-6,9,12,15-octadecatetraenoic acid |
| Eicosatrienoic acid (ETE) | 20:3 (n-3) | all-cis-11,14,17-eicosatrienoic acid |
| Eicosatetraenoic acid (ETA) | 20:4 (n-3) | all-cis-8,11,14,17-eicosatetraenoic acid |
| Eicosapentaenoic acid (EPA) | 20:5 (n-3) | all-cis-5,8,11,14,17-eicosapentaenoic acid |
| Heneicosapentaenoic acid (HPA) | 21:5 (n-3) | all-cis-6,9,12,15,18-heneicosapentaenoic acid |
| Docosapentaenoic acid (DPA), | 22:5 (n-3) | all-cis-7,10,13,16,19-docosapentaenoic acid |
| Clupanodonic acid | | |
| Docosahexaenoic acid (DHA) | 22:6 (n-3) | all-cis-4,7,10,13,16,19-docosahexaenoic acid |
| Tetracosapentaenoic acid | 24:5 (n-3) | all-cis-9,12,15,18,21-tetracosapentaenoic acid |
| Tetracosahexaenoic acid (Nisinic acid) | 24:6 (n-3) | all-cis-6,9,12,15,18,21-tetracosahexaenoic acid |
| Linoleic acid (LA) | 18:2 (n-6) | all-cis-9,12-octadecadienoic acid |
| Gamma-linolenic acid (GLA) | 18:3 (n-6) | all-cis-6,9,12-octadecatrienoic acid |
| Calendic acid | 18:3 (n-6) | 8E,10E,12Z-octadecatrienoic acid |
| Eicosadienoic acid | 20:2 (n-6) | all-cis-11,14-eicosadienoic acid |
| Dihomo-gamma-linolenic acid (DGLA) | 20:3 (n-6) | all-cis-8,11,14-eicosatrienoic acid |
| Arachidonic acid (AA) | 20:4 (n-6) | all-cis-5,8,11,14-eicosatetraenoic acid |
| Docosadienoic acid | 22:2 (n-6) | all-cis-13,16-docosadienoic acid |
| Adrenic acid | 22:4 (n-6) | all-cis-7,10,13,16-docosatetraenoic acid |
| Docosapentaenoic acid | 22:5 (n-6) | all-cis-4,7,10,13,16-docosapentaenoic acid |
| Tetracosatetraenoic acid | 24:4 (n-6) | all-cis-9,12,15,18-tetracosatetraenoic acid |
| Tetracosapentaenoic acid | 24:5 (n-6) | all-cis-6,9,12,15,18-tetracosapentaenoic acid |
| None | 12:1 (n-7) | 5-Dodecenoic acid |
| None | 14:1 (n-7) | 7-Tetradecenoic acid |
| Palmitoleic acid | 16:1 (n-7) | 9-Hexadecenoic acid |
| Vaccenic acid | 18:1 (n-7) | 11-Octadecenoic acid |
| Paullinic acid | 20:1 (n-7) | 13-Eicosenoic acid |
| None | 22:1 (n-7) | 15-Docosenoic acid |
| None | 24:1 (n-7) | 17-Tetracosenoic acid |
| oleic acid | 18:1 (n-9) | 9-octadecenoic acid |
| elaidic acid | 18:1 (n-9) | (E)-octadec-9-enoic acid |
| gondoic acid | 20:1 (n-9) | 11-eicosenoic acid |
| mead acid | 20:3 (n-9) | 5,8,11-eicosatrienoic acid |
| erucic acid | 22:1 (n-9) | 13-docosenoic acid |
| nervonic acid | 24:1 (n-9) | 15-tetracosenoic acid |

Conjugated Linoleic Acids (two conjugated double bonds)

| | | |
|---|---|---|
| Rumenic acid | 18:2 (n-7) | 9Z,11E-octadeca-9,11-dienoic acid |
| | 18:2 (n-6) | 10E,12Z-octadeca-9,11-dienoic acid |

Conjugated Linolenic Acids (three conjugated double bonds)

| | | |
|---|---|---|
| α-Calendic acid | 18:3 (n-6) | 8E,10E,12Z-octadecatrienoic acid |
| β-Calendic acid | 18:3 (n-6) | 8E,10E,12E-octadecatrienoic acid |
| Jacaric acid | 18:3 (n-6) | 8Z,10E,12Z-octadecatrienoic acid |
| α-Eleostearic acid | 18:3 (n-5) | 9Z,11E,13E-octadeca-9,11,13-trienoic acid |
| β-Eleostearic acid | 18:3 (n-5) | 9E,11E,13E-octadeca-9,11,13-trienoic acid |
| Catalpic acid | 18:3 (n-5) | 9Z,11Z,13E-octadeca-9,11,13-trienoic acid |
| Punicic acid | 18:3 (n-5) | 9Z,11E,13Z-octadeca-9,11,13-trienoic acid |

Other

| | | |
|---|---|---|
| Rumelenic acid | 18:3 (n-3) | 9E,11Z,15E-octadeca-9,11,15-trienoic acid |
| α-Parinaric acid | 18:4 (n-3) | 9E,11Z,13Z,15E-octadeca-9,11,13,15-tetraenoic acid |
| β-Parinaric acid | 18:4 (n-3) | all trans-octadeca-9,11,13,15-tretraenoic acid |
| Bosseopentaenoic acid | 20:5 (n-6) | 5Z,8Z,10E,12E,14Z-eicosanoic acid |
| Pinolenic acid | 18:3 (n-6) | (5Z,9Z,12Z)-octadeca-5,9,12-trienoic acid |
| Podocarpic acid | 20:3 (n-6) | (5Z,11Z,14Z)-eicosa-5,11,14-trienoic acid |

The omega-3 and omega-6 fatty acids are commonly referred to as “essential” fatty acids because the human or animal body cannot synthesize them and therefore they must be obtained from food or other dietary sources. In one embodiment, the fatty acid component comprises or consists of essential fatty acids.

In embodiments where the fatty acid component is an omega-3 fatty acid, the omega-3 fatty acid may be selected from the group consisting of hexadecatrienoic acid (HTA), alpha-linolenic acid (ALA), stearidonic acid (SDA), eicosatrienoic acid (ETE), eicosatetraenoic acid (ETA), eicosapentaenoic acid (EPA, timnodonic acid), heneicosapentaenoic acid (HPA), docosapentaenoic acid (DPA, clupanodonic acid), docosahexaenoic acid (DHA, Cervonic acid), tetracosapentaenoic acid, 24:5 (n-3), and tetracosahexaenoic acid (Nisinic acid), 24:6 (n-3).

In embodiments where the fatty acid component is an omega-6 fatty acid, the omega-6 fatty acid may be selected from the group consisting of linoleic acid (LA), gamma-linolenic acid (GLA), eicosadienoic acid, dihomo-gamma-linolenic acid (DGLA), arachidonic acid (AA), docosadienoic acid, adrenic acid, docosapentaenoic acid (Osbond acid), tetracosatetraenoic acid, and tetracosapentaenoic acid, 24:5 (n-6).

In embodiments where the fatty acid component is an omega-9 fatty acid, the omega-9 fatty acid may be selected from the group consisting of mead acid, 20:3 (n-9), all-cis-5,8,11-eicosatrienoic acid. In one embodiment, the monounsaturated omega-9 fatty acid is selected from the group consisting of oleic acid, eicosenoic acid, erucic acid, and nervonic acid.

It is noted that docosapentaenoic acid exists as two separate analogs and each analog is in a separate fatty acid series, either the omega-3 or omega-6 series. Both compounds have the same empirical formula and molecular weight, and both have five all cis double bonds, but each differs in the position those double bonds occupy in the 22 carbon, long chain fatty acid. The omega-3 analog is commonly referred to as DPA, docosapentaenoic acid or clupanodonic acid and is all-cis-7,10,13,16,19-docosapentaenoic acid. The omega-6 analog is commonly called Osbond acid and chemically is all-cis-4,7,10,13,16-docosapentaenoic acid. Accordingly, where docosapentaenoic acid is referred to as an omega-3 fatty acid herein, all-cis-7,10,13,16,19-docosapentaenoic acid is intended (DPA or clupanodonic acid), and where docosapentaenoic acid is referred to as an omega-6 fatty acid herein, all-cis-4,7,10,13,16-docosapentaenoic acid (Osbond acid) is intended.

### Physical Properties

Generally, the compounds of the disclosure are physically and chemically stable solid materials, e.g., powders. In embodiments, the compounds are solid, free flowing powders suitable for formulation into solid dosage forms such as powders, tablets, capsules or caplets. The solid, free-flowing character of the compounds also provides for ease of their formulation in physical admixture with each other and with other active agents in the same solid dosage form. In embodiments, the compounds are non-hygroscopic or have low hygroscopicity. In embodiments, the compounds are highly water soluble.

The compounds of the disclosure generally possess superior chemical and physical stability of the fatty acid component, for example as compared to fatty acid compounds based upon the oil form of the fatty acid, e.g., the free fatty acids or the ethyl ester or glyceryl ester forms of the fatty acids. Since the compounds described here are solids (not oils) they are very stable against oxidative degradation of the fatty acid component, particularly when compared to the free fatty acid or fatty acid ester forms of the fatty acids, which are highly susceptible to oxidative degradation in their liquid forms and consequently tend to degrade when exposed to air or humidity. In contrast, the compounds described here, are, for example, relatively more stable to air, oxygen, and humidity compared to compounds and compositions comprising the free fatty acids or the ethyl ester or glyceryl ester forms of the fatty acids.

The compounds described here are also expected to provide improved bioavailability of the fatty acid component, due to improved pharmacokinetics, compared to other dosage forms comprising, for example, the oily free fatty acids and esters of the fatty acids.

### Compositions

The present disclosure provides compositions comprising one or more of the compounds described herein, including compositions comprising mixtures of two or more different compounds described herein. Also provided are compositions comprising one or more of the compounds described herein, or mixtures of same, along with a second active agent. In embodiments the second active agent is a biologically active agent or an active pharmaceutical ingredient (API).

The compositions comprising one or more compounds of the invention may be formulated as a solid dosage form selected from a powder, tablet, capsule, or caplet. In one embodiment, the solid dosage form is adapted for oral delivery. In one embodiment, the solid dosage form is adapted for once a day delivery. In another embodiment, the solid dosage form is adapted for delivery twice a day. In one embodiment, the dosage form is an oral dosage form. The oral dosage form may be in the form of a solid, such as a tablet, a capsule containing particulates, liquids, or powders, a lozenge (including liquid-filled), a gum, or a gel. In one embodiment, the dosage form is a solid oral dosage form.

In one embodiment, the composition is a powder suitable for reconstitution in an aqueous liquid. Such powders may be used, for example, to prepare a liquid suitable for intraperitoneal delivery of the compounds described here.

In one embodiment, the composition is a pharmaceutical composition and the carrier is acceptable for administration to humans or non-human animals, as described in more detail infra.

In one embodiment, the composition is a dietary supplement or additive and the carrier is acceptable for administration to humans or non-human animals, as described in more detail infra.

A composition may be in the form of a unit dose. The unit dose may be, for example, in the form of a tablet or capsule. In one embodiment, the composition comprises a compound of Formula I or II. In one embodiment, a unit dose of the composition contains from about 0.05 g to 12 g of total fatty acids. In one embodiment, the unit dose contains from about 0.05 g, 1 g, 2 g, 3 g, 4 g, 5 g, or 6 g of total fatty acids.

In embodiments, a composition of the invention contains from about 50 mg to 6 g of fatty acids in the fatty acid counter ion component. In one embodiment, a unit dose of such a composition comprises from about 50 mg to 6 g, or from about 500 mg to 6 g, or at least 200 mg, at least 300 mg, at least 400 mg, at least 500 mg, or at least 1 g of fatty acids, especially or particularly polyunsaturated fatty acids. In one embodiment, the fatty acid component of the composition consists of at least 70%, at least 80%, or at least 90% by weight of one or more polyunsaturated fatty acids, or from about 20% to 90%, from 30% to 90%, from 40% to 90%, from 50% to 90%, from 60% to 90%, or from 70% to 90% by weight of one or more PUFAs. In one embodiment, the fatty acids are selected from two or more of EPA, DHA, DPA, hexadecatrienoic acid (HTA), linoleic acid (LA), □-linolenic acid (GLA), α-linolenic acid (ALA), stearidonic acid (SDA), eicosadienoic acid, eicosatrienoic acid (ETE), eicosatetraenoic acid (ETA), heneicosapentaenoic acid (HPA), tetracosapentaenoic acid, tetracosatetraenoic acid, tetracosahexaenoic acid, calendic acid, eicosadienoic acid, dihomogamma-linolenic acid (DGLA), arachidonic acid (AA), docosadienoic acid, adrenic acid, Osbond acid, palmitoleic acid, vaccenic acid, paullinic acid, oleic acid, elaidic acid, gondoic acid, mead acid, erucic acid, and nervonic acid. In one embodiment, the fatty acids are selected from two or more of EPA, DHA, and DPA.

The compounds described here may be formulated alone or in combination with one or more additional active pharmaceutical ingredients (API) or biologically active agents. In one embodiment, a compound described here is formulated with one or more additional APIs or biologically active agents in a single dosage form. In one embodiment, the dosage form is a solid dosage form. In one embodiment, the solid dosage form is a powder suitable for reconstitution in aqueous media.

Depending on the nature of the compounds and excipients making up the composition, the composition may be suitable for pharmaceutical or veterinary use, or for use a dietary additive or supplement, or any combination of these uses. The various compositions are discussed in the following sections as “pharmaceutical compositions” and “additives and supplements” but these terms are not meant to be limiting, only descriptive.

The compositions of the invention may be formulated using one or more suitable excipients or carriers. A suitable excipient or carrier is one suitable for human or animal use. The term “excipient” refers to an additive that serves some purpose in the composition other than a carrier, for example as a stabilizer, taste masking agent (e.g., a sweetener), solubilizing agent, or suspending agent. Often, a carrier will serve a dual purpose as a simple carrier or diluent and an excipient. Examples of pharmaceutically acceptable excipients may thus include carriers. Non-limiting examples of excipients for use in the compositions of the invention include sterile liquids, water, buffered saline, ethanol, polyols (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, and suitable mixtures thereof.

A suitable excipient or carrier is typically a pharmaceutically acceptable carrier or excipient for use in animals or humans (or both). The term “pharmaceutically acceptable” indicates approval by a regulatory agency of the Federal or a state government or listed in the U.S. Pharmacopeia or other generally recognized pharmacopeia such as the European Pharmacopeia, for use in animals, and more particularly in humans. In the context of the pharmaceutical compositions of the invention, a “carrier” refers to, for example, a solvent, a diluent, or vehicle with which the ionic salt of the invention is formulated for delivery. Examples of pharmaceutically acceptable carriers for use in the compositions of the invention include, without limitation, sterile aqueous and non-aqueous liquids, water, buffered saline, ethanol, polyols (for example, glycerol, propylene glycol, liquid polyethylene glycol and the like), and oils, for liquid dosage forms; or carbohydrates (e.g., glucose, lactose, sucrose or dextran) for solid dosage forms.

The compounds of the invention may be formulated in any suitable form and for any suitable intended route of administration. Typically, the dosage form is at least in part determined by the intended route of administration.

In one embodiment, the dosage form is a liquid suitable for administration to the eye. The formulation may be a solution, suspension, or gel suitable for ocular administration, e.g., suitable for topical administration to the eye, also referred to as an ophthalmic formulation.

In one embodiment, the ophthalmic formulation is an aqueous formulation. In one embodiment, the ophthalmic formulation comprises one or more of glycerin, hypromellose, propylene glycol or polyethylene glycol. In one embodiment, the ophthalmic formulation further comprises one or more of polysorbate 80, carbomer copolymer type A, purified water, sodium hydroxide, ascorbic acid, benzalkonium chloride, boric acid, dextrose, disodium phosphate, glycine, magnesium chloride, potassium chloride, sodium borate, sodium chloride, sodium citrate, sodium lactate, edetate disodium, hydrochloric acid, sodium hydroxide, aminornethylpropanol, hydroxypropyl guar, polyquaternium-I, or sorbitol.

In one embodiment, the ophthalmic formulation comprises one or more of surfactants, tonicity agents, buffers, preservatives, co-solvents and viscosity building agents. Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Preferably, the tonicity agent is present in an amount sufficient to cause the final composition to have an ophthalmically acceptable osmolality (generally about 150-450 mOsm, preferably 250-350 mOsm). An appropriate buffer system (e.g., sodium phosphate, sodium acetate, sodium citrate, sodium borate or boric acid) may be added to the compositions to prevent pH drift under storage conditions. The particular concentration will vary, depending on the agent employed. Preferably, however, the buffer will be chosen to maintain a target pH within the range of pH 6-7.5.

Compositions formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous compositions which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of one or more of the fatty acid salts of the invention.

Examples or artificial tears compositions useful as artificial tears carriers include, but are not limited to, commercial products, such as Tears Naturale™, Tears Naturale n™, Tears Naturale Free™, and Bion Tears™. (Alcon Laboratories, Inc., Fort Worth, Tex.). Examples of phospholipid carrier formulations include those disclosed in U.S. Pat. No. 4,804,539 (Guo et al.), U.S. Pat. No. 4,883,658 (Holly), U.S. Pat. No. 4,914,088 (Glonek), U.S. Pat. No. 5,075,104 (Gressel et al.), U.S. Pat. No. 5,278,151 (Korb et al.), U.S. Pat. No. 5,294,607 (Glonek et al.), U.S. Pat. No. 5,371,108 (Korb et al.), U.S. Pat. No. 5,578,586 (Gionek et al.); the foregoing patents are incorporated herein by reference to the extent they disclose phospholipid compositions useful as phospholipid carriers of the present invention.

Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as polyvinyl alcohol, polyvinylpyrrolidone, povidone and carbomers, such as carbomer 934P, carbomer 941, carbomer 940, carbomer 974P.

Examples of viscosity enhancing agents include, but are not limited to polysaccharides, such as hyaluronic acid and its salts, chondroitin sulfate and its salts, dextrans, various polymers of the cellulose family; vinyl polymers; and acrylic acid polymers. In general, the phospholipid carrier or artificial tears will exhibit a viscosity of 1 to 400 centipoises (“cps”). Topical ophthalmic products are typically packaged in multidose form. Preservatives may be required to prevent microbial contamination during use. Suitable preservatives include benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Other wetting agents, emulsifiers and lubricants, such as sodium lauryl sulfate and magnesium stearate, as well as coloring agents, release agents, coating agents, and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, a-tocopherol, and the like; and metal chelating agents, such as citric acid, ethylenediamine tetraacetic acid (EDTA), sorbitol, tartaric acid, phosphoric acid, and the like.

A contact lens may optionally be used to allow for extravasation of vasoactive substance over a more prolonged time period. Vasoactive substances such as Thrombin and Thromboxane A may further induce increase in tear volume via venular vasoconstriction and increased perfusion through lacrimal, accessory lacrimal and surface microvessels; where increased paracellular endothelial openings that increase capillary permeability can further enhance this benefit.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers.

### Pharmaceutical Compositions

In embodiments, the composition is a pharmaceutical composition comprising a compound of Formula I or Formula II, and optionally a pharmaceutically acceptable carrier and/or excipient. In embodiments, the composition further comprises an additional active agent, such as an API, as described below.

In embodiments is provided a solid dosage form comprising a compound of the invention in physical admixture with one or more additional active pharmaceutical ingredients (APIs). In embodiments, the one or more additional APIs is an antihyperlipidemic agent, an anti-diabetic agent, an anti-epileptic agent, or an anti-inflammatory agent.

In embodiments, the anti-inflammatory agent is an ICAM-1 antisense oligonucleotide (e.g., ISIS-3082), MCP-1, or an NSAID, or a combination thereof. In embodiments, the anti-inflammatory agent is an NSAID.

In embodiments the API is an antihyperlipidemic agent or an anti-diabetic agent. In embodiments, the antihyperlipidemic agent is selected from the group consisting of an HMG CoA enzyme inhibitor (e.g., a statin), a cholesterol absorption inhibitor, and a cholesterol esterase transfer protein (CETP) inhibitor. In one embodiment, the antihyperlipidemic agent is a statin. In one embodiment, the statin is selected from the group consisting of atorvastatin, risuvostatin, simvastatin, pravastatin, and pharmaceutically acceptable salts or prodrugs thereof. In one embodiment, the statin is present in an amount ranging from 5 mg to 100 mg. In one embodiment, the statin is pravastatin. In one embodiment, the antihyperlipidemic agent is a cholesterol absorption inhibitor. In one embodiment, the cholesterol absorption inhibitor is ezetimibe, also known as Zetia. In one embodiment, the antihyperlipidemic agent is a CETP inhibitor. In one embodiment, the CETP inhibitor is anacetrapib, or a hydrate, or solvate thereof.

In one embodiment, the pharmaceutical composition comprises an amount of one or more of the compounds described here effective to lower elevated serum triglycerides in a subject, preferably a human subject. In one embodiment, the subject is a human subject having severe hypertriglyceridemia. In one embodiment, the subject is a human subject having non-severe hypertriglyceridemia.

In embodiments, the pharmaceutical composition comprises an amount of one or more of the compounds described here effective to treat a metabolic disorder selected from the group consisting of abnormal glucose metabolism manifesting in diabetes or pre-diabetes, abnormal lipid metabolism manifesting as hypertriglyceridemia, i.e., elevated triglycerides, mixed dyslipidemia, fatty liver, and combined abnormal glucose and lipid metabolism manifesting in obesity. In one embodiment, a composition of the invention is used in a method for treating a disease or disorder selected from diabetes, pre-diabetes, hypertriglyceridemia, dyslipidemia, fatty liver, and obesity.

In embodiments, the pharmaceutical composition comprises an amount of one or more of the compounds described here effective to treat a disease or disorder selected from the group consisting of arthritis, irritable bowel syndrome, atrial fibrillation, ophthalmic inflammation disorders, dry eye syndrome, traumatic brain injury, familial adenomatous polyposis, sporadic adenomatous polyposis, epilepsy, epileptic syndrome, Alzheimer's disease, and attention deficit hyperactivity disorder (ADHD).

In embodiments, the pharmaceutical composition comprises an amount of one or more of the compounds described here effective to treat a gastrointestinal inflammatory disease or disorder in a subject where the disease or disorder is selected from postoperative intestinal inflammation, postoperative ileus, ischemia reperfusion injury, or a combination thereof. In embodiments, the gastrointestinal inflammatory disease or disorder is post-operative ileus (POI).

In one embodiment, the pharmaceutical composition comprises an amount of one or more of the compounds of Formula I effective to treat or manage pain in a subject. In one embodiment, the pain is neuropathic pain or nociceptive pain. In embodiments, the method is for treating nociceptive pain, the method comprising administering to a subject in need of such treatment, a composition comprising a compound of Formula I and gabapentin. In embodiments, the disclosure provides a method for treating neuropathic pain, the method comprising administering to a subject in need of such treatment, a composition comprising a compound of Formula I and an NSAID. In embodiments, the fatty acid component of the compound of Formula I is an omega-3 fatty acid, preferably selected from eicosapentaenoic acid (EPA), docosahexaenoic acid (DHA), and docosapentaenoic acid (DPA).

Particular compounds for use in treating the various diseases and disorders referred to here are described in more detail infra.

### Non-Pharmaceutical Compositions

The compounds of the invention may also be formulated with one or more additional non-pharmaceutical agents, for example beneficial biologically active agents, such as a nutrient or nutraceutical compounds, including e.g., vitamins, minerals, botanical extracts, etc., in the same dosage form, along with any suitable excipients or carriers. In one embodiment, the one or more additional biologically active agents is selected from the group consisting of a vitamin, a mineral, an amino acid, a carbohydrate, an antioxidant, a flavonoid, a carotenoid, a phytoseterol, an herb, an enzyme, a botanical extract or concentrate, and a botanical compound. In one embodiment, the one or more additional biologically active agents is selected from the group consisting of vitamin A, vitamin B1, vitamin B12, vitamin B6, vitamin C, vitamin D, vitamin E, vitamin K, calcium, carnitine, chromium, chondroitin, coenzyme Q10 (ubiquinone), folate, glucosamine, metafolin, riboflavin, biotin, iodine, iron, magnesium, selenium, thiamin, and zinc. In one embodiment, the one or more additional biologically active agents is selected from the group consisting of coenzyme Q10, L-carnitine, an antioxidant, a phytoseterol, and a flavonoid. In one embodiment, the antioxidant is a polyphenol. In one embodiment, the polyphenol is selected from lycopene, resveratrol, and epigallocatechingallate.

In one embodiment, a compound described here is useful as a dietary supplement or nutraceutical additive. In an embodiment, a compound of Formula I is formulated as a nutraceutical additive or supplement, either alone or in combination with one or more additives or supplements and any suitable excipients. In one embodiment, the nutraceutical additive or supplement is in the form of a powder. In one embodiment, the nutraceutical additive or supplement is in the form of a liquid. In one embodiment, the nutraceutical additive or supplement is in the form of a mouth wash, a dentifrice, chewing gum, a candy, a tablet, a capsule, a mouth spray, or a film.

In one embodiment, the nutraceutical additive forms part of a food or drink product suitable for human consumption. There is no specific limitation on the foods/drinks to which a nutraceutical additive of the invention can be incorporated. Examples of such foods/drinks include processed foods based on meat, poultry meat, fish/shellfish and the like; soup; seasonings including sweetener and the like; rice seasonings; instant foods; frozen foods; snacks; various types of functional foods such as supplements, nutritional drinks and the like; canned foods; dairy products; confectionery such as chewing gum, candy, gummy candy, chocolate, baked sweets and the like; ice cream; soft drinks such as tea, coffee, cocoa, fruit juice, sports drink, carbonated drink, vegetable drink and the like; liquors; soya milk; lactic acid bacteria beverages; and chlorophyll juice.

The amount of the nutraceutical additive of the invention incorporated into the food or drink varies in accordance with the type of food or drink and the amount that one wishes to supplement a diet with one or more omega-3 fatty acids. In one embodiment, the nutraceutical additive is incorporated into the food or drink so as to provide an amount of the omega-3 fatty acid that is about 0.000001 to 20% by weight, based on total weight of the food or drink product, and more preferably in an amount of about 0.00001 to 10% by weight.

### Methods of Use

The compositions of the invention are useful in methods of treating various diseases and disorders that are responsive to treatment with fatty acids. In addition, the compositions of the invention may have non-pharmaceutical uses, for example as dietary supplements or additives. These uses are described in more detail infra.

In one embodiment, the methods relate to diseases and disorders that are responsive to treatment with fatty acids, especially polyunsaturated fatty acids, and particularly polyunsaturated fatty acids of the omega-3, omega-6, omega-7, and omega-9 series. The methods relating to diseases or disorders that are responsive to treatment with fatty acids, are discussed in more detail infra.

In accordance with any of the following embodiments the fatty acid component of a compound described herein may be selected from a fatty acid of the omega-3, omega-6, omega-7, or omega-9 series, including any of the fatty acids set forth in Table 1, and combinations thereof, as described supra. In one embodiment, at least 50%, at least 60%, at least 70%, at least 80%, or at least 90% of the fatty acid component consists of one or more omega-3 fatty acids independently selected from eicosapentaenoic acid (EPA), docosahexaenoic acid (DHA), and docosapentaenoic acid (DPA).

In embodiments, the fatty acid component of the compounds administered according to the methods described herein comprises a long-chain polyunsaturated fatty acid, a medium chain polyunsaturated fatty acid, or a mixture of medium and long chain polyunsaturated fatty acids. In embodiments, the polyunsaturated fatty acids are selected from a fatty acid of the omega-3, omega-6, omega-7, or omega-9 series. In embodiments, the polyunsaturated fatty acids are omega-3 fatty acids selected from eicosapentaenoic acid (EPA), docosahexaenoic acid (DHA), and docosapentaenoic acid (DPA). In embodiments, at least 50% of the fatty acid component of the composition consists of one or more omega-3 fatty acids selected from eicosapentaenoic acid (EPA), docosahexaenoic acid (DHA), and docosapentaenoic acid (DPA).

In the context of any of the methods described here, a compound described herein may be formulated as a pharmaceutical composition, or as a food additive or supplement, meaning that the compound itself and any additives or excipients in the formulation are suitable for administration to humans or animals.

In the context of the methods described here, the term “treating” may refer to the amelioration or stabilization of one or more symptoms associated with the disease or disorder being treated. The term “treating” may also encompass the management of a disease or disorder, referring to the beneficial effects that a subject derives from a therapy which does not result in a cure of the underlying disease or disorder. For example, lowering elevated plasma triglycerides can be considered an aspect of treating diabetes because it is a beneficial effect that does not result in a cure of the underlying defect of glucose metabolism. The compositions of the invention can also be used in the prevention of certain diseases, disorders, and conditions. In this context, the term “prevention” refers to preventing the recurrence, development, progression or onset of one or more symptoms of the disease, disorder, or condition.

In accordance with the methods described here, a therapeutically effective amount of a compound described herein is administered to a subject, the therapeutically effective amount being an amount of the compound (or mixture of two or more compounds) sufficient to achieve a desired therapeutic outcome, for example the amelioration or stabilization of one or more symptoms of the disease or disorder being treated, or in the context of prevention, the amount sufficient to achieve prevention of the recurrence, development, progression or onset of one or more symptoms of the disease, disorder, or condition.

For administration to human patients, the total daily dose of a compound of the invention is typically in the range 50 mg to 12 g depending, of course, on the route of administration. In one embodiment the total daily dose is in the range of from about 100 mg to 500 mg, about 500 mg to 1 g, about 1 g to 2 g, about 2 g to 5 g, or about 5 g to 10 g. In another embodiment the total daily dose is in the range 4 g to 8 g and in yet another embodiment the total daily dose is in the range 1 g to 2 g. The total daily dose may be administered in single or divided doses.

These dosages are based on an average human subject having a weight of about 65 kg to 70 kg. The physician will readily be able to determine doses for subjects whose weight falls outside this range, such as infants and the elderly.

In one embodiment, a therapeutically effective amount is the amount required to achieve at least an equivalent therapeutic effect compared to a standard therapy. An example of a standard therapy is an FDA-approved drug indicated for treating a particular disease or disorder. As a concrete example, Vascepa™ is an FDA-approved formulation of EPA, specifically an ethyl ester of EPA. Accordingly, in one aspect, the methods of the invention include administering to a subject a therapeutically effective amount of a compound of Formula IA or IB or a composition comprising same, or a composition comprising mixtures of at least two different compounds of Formula IA, as described herein, which is effective to reduce plasma triglycerides in an adult human subject by at least about 1 mmol/L, or by at least about 2 mmol/L.

In the context of any of the methods of the present invention, the subject may be a human or a non-human mammal. The non-human mammal may be, for example, a non-human primate, a dog, cat, a rodent (e.g., a mouse, a rat, a rabbit), a horse, a cow, a sheep, a goat, a bird, a chicken, or any other non-human mammal Preferably, the subject is a human.

In one embodiment, the subject is a human subject. In one embodiment, the human is an adult human, a pediatric human, or a geriatric human, as those terms are understood by the medical practitioner, for example as defined by the U.S. Food and Drug Administration.

The compounds or compositions described here can be used as monotherapy or adjunctive therapy. The compositions of the invention can be administered alone or in combination with one or more additional therapeutic agents (i.e., additional APIs) or therapies, for example as part of a therapeutic regimen that includes, e.g., aspects of diet and exercise. In certain embodiments, the methods of the invention include administration of a composition of the invention as the primary therapy. In other embodiments, the administration of a composition of the invention is an adjuvant therapy. In either case, the methods of the invention contemplate the administration of a composition of the invention in combination with one or more additional therapeutic agents and/or therapies for the treatment or prevention of a disease or disorder. The terms “therapy” and “therapies” refer to any method, protocol and/or agent that can be used in the prevention, treatment, management or amelioration of a disease or disorder, or one or more symptoms thereof.

The compounds or compositions described here can also be used in combination therapy. As used herein, “combination therapy” or “co-therapy” includes the administration of a therapeutically effective amount of one or more of the compounds described here as part of a specific treatment regimen intended to provide the beneficial effect from the co-action of the one or more compounds and an additional active agent, for example an additional API or active biological agent as described above. The beneficial effect of the combination includes, but is not limited to, pharmacokinetic or pharmacodynamic co-action resulting from the combination. The beneficial effect of the combination may also relate to the mitigation of a toxicity, side effect, or adverse event associated with another agent in the combination. “Combination therapy” is not intended to encompass the administration of two or more compounds as part of separate monotherapy regimens that incidentally and arbitrarily result in a beneficial effect that was not intended or predicted.

### Metabolic Disorders

In one embodiment, the invention provides methods of treating a metabolic disorder in a subject in need thereof, the method comprising administering to the subject, preferably a human subject, an effective amount of a composition comprising a compound of Formula I or Formula II, or mixtures thereof.

In one embodiment the metabolic disorder is selected from the group consisting of abnormal glucose metabolism manifesting in diabetes or pre-diabetes, abnormal lipid metabolism manifesting as hypertriglyceridemia, i.e., elevated triglycerides, mixed dyslipidemia, hypercholesterolemia, fatty liver, and combined abnormal glucose and lipid metabolism manifesting in obesity. In one embodiment the metabolic disorder is a dyslipidemic disorder selected from hypertriglyceridemia, hypercholesterolemia and mixed dyslipidemias. In one embodiment, the metabolic disorder is selected from the group consisting of pre-diabetes, type 2 diabetes, obesity, fatty liver disease, and insulin resistance.

In one embodiment, the methods comprise administering a therapeutically effective amount, which amount is effective to reduce plasma triglycerides in an adult human subject by at least about 0.5 mmol/L, about 1 mmol/L, or about 2 mmol/L.

In one embodiment, the subject is a human subject having severe hypertriglyceridemia characterized by serum triglyceride levels of from 500 to 2,000 mg/dl.

### Cardiovascular Disorders

In one embodiment, the invention provides a method for treating cardiovascular disorders or complications relating to atrial fibrillation, myocardial infarction, and congestive heart failure by administering to a subject in need of such treatment an effective amount of a composition comprising a compound of Formula I, or mixtures thereof.

In embodiments, at least 50% of the fatty acid component of the composition consists of one or more omega-3 fatty acids selected from eicosapentaenoic acid (EPA), docosahexaenoic acid (DHA), and docosapentaenoic acid (DPA).

In one embodiment, the effective amount is effective to treat one or more symptoms of the cardiovascular condition

### Hematological Disorders

In one embodiment, the invention provides a method for treating hematological disorders or complications relating to sickle cell disease by administering to a subject in need of such treatment an effective amount of a composition comprising a compound of Formula I, or mixtures thereof.

In one embodiment, the effective amount is effective to treat one or more symptoms of the sickle cell disease.

### Cancer Treatment and Prevention

In one embodiment, the invention provides a method for preventing cancer, the method comprising administering a therapeutically effective amount of a composition comprising a compound of Formula I, or mixtures thereof. In one embodiment, the cancer is colon cancer or familial adenomatous polyposis.

### Inflammatory Disorders

The compounds of Formula I, or mixtures thereof, may be particularly useful in the treatment of diseases and disorders having a significant inflammatory component, due to the anti-inflammatory properties of polyunsaturated fatty acids and the ability of the compounds of Formula I to deliver high amounts of free fatty acids to the serum by oral routes of administration.

In one embodiment, the invention provides a method for treating an inflammatory disorder, the method comprising administering to a subject in need of such treatment an effective amount of a composition comprising a compound of Formula I, or mixtures thereof. In one embodiment, the effective amount is effective to treat one or more symptoms of the inflammatory disorder. In one embodiment, the inflammatory disorder is selected from the group consisting of arthritis, inflammatory bowel disease, and psoriasis.

In one embodiment, the invention provides methods of treating arthritis, irritable bowel syndrome, ophthalmic inflammation disorders, or dry eye syndrome in a subject in need of such treatment, the methods comprising administering to the subject a composition comprising a compound of Formula I, or mixtures thereof.

In one embodiment, the invention provides methods of treating a gastrointestinal inflammatory disease or disorder in a subject in need thereof, the method comprising administering to the subject, preferably a human subject, an effective amount of a composition comprising a compound of Formula I, or mixtures thereof. In embodiments, the gastrointestinal inflammatory disease or disorder is postoperative intestinal inflammation, postoperative ileus, ischemia reperfusion injury, or a combination thereof. In one embodiment, the gastrointestinal inflammatory disease or disorder is post-operative ileus (POI). In embodiments, the method comprises administering a compound described herein with an anti-inflammatory agent. In embodiments, the compound and the anti-inflammatory agent are contained in the same dosage form.

In one embodiment, the invention provides a method for treating a disease or disorder of the ocular system, also referred to as ophthalmic diseases and disorders, having an underlying inflammatory component, the method comprising administering to a subject in need of such treatment an effective amount of a composition comprising a compound of Formula I, or mixtures thereof.

In one embodiment, at least 50% of the fatty acid component of the composition consists of one or more omega-3 fatty acids independently selected from eicosapentaenoic acid (EPA), docosahexaenoic acid (DHA), and docosapentaenoic acid (DPA).

In one embodiment, the effective amount is effective to treat one or more symptoms of the disease or disorder of the ocular system. In one embodiment, the disease or disorder of the ocular system is selected from the group consisting of inflammatory diseases of the eye, dry eye syndrome, macular edema and retinopathy. In one embodiment, the method is a method for promoting corneal wound healing.

In one embodiment, the invention provides a method for treating dry eye by administering a composition comprising a compound of Formula I, or mixtures thereof.

In one embodiment, at least 50% of the fatty acid component of the composition consists of one or more omega-3 fatty acids independently selected from eicosapentaenoic acid (EPA), docosahexaenoic acid (DHA), and docosapentaenoic acid (DPA).

Dry eye disease or syndrome is a multifactorial disorder of the tears and ocular surface characterized by symptoms of dryness and irritation. Inflammation is an important component in the development and propagation of dry eye (Stevenson et al., Arch. Ophthalmol., 2012, 130(1),90-100; Rashid et al., Arch. Ophthalmol., 2008, 126(2),219-225).

The term ‘dry eye’ refers to inadequate tear production and/or abnormal tear composition. Causes of dry eye disease as defined herein include but are not limited to the following: idiopathic, congenital alacrima, xerophthalmia, lacrimal gland ablation, and sensory denervation; collagen vascular diseases, including rheumatoid arthritis, Wegener's granulomatosis, and systemic lupus erythematosus; Sjogren's syndrome and autoimmune diseases associated with Sjogren's syndrome; abnormalities of the lipid tear layer caused by blepharitis or rosacea; abnormalities of the mucin tear layer caused by vitamin A deficiency; trachoma, diphtheric keratoconjunctivitis; mucocutaneous disorders; aging; menopause; and diabetes. Further, the term “dry eye” includes dry eye after anterior ophthalmic operation such as cataract operation and refractive surgery and that accompanied with allergic conjunctivitis Dry eye symptoms as defined herein may also be provoked by other circumstances, including, but not limited to, the following: prolonged visual tasking; working on a computer; being in a dry environment; ocular irritation; contact lenses, LASIK and other refractive surgeries; fatigue; and medications such as isotretinoin, sedatives, diuretics, tricyclic antidepressants, antihypertensives, oral contraceptives, antihistamines, nasal decongestants, beta-blockers, phenothiazines, atropine, and pain relieving opiates such as morphine.

### Neurological Disorders

In one embodiment, the invention provides a method for treating a psychiatric disorder in a subject, the method comprising administering the subject a therapeutically effective amount of a composition comprising a compound of Formula I, or mixtures thereof, where the amount is effective to treat one or more symptoms of the psychiatric disorder.

In embodiments, at least 50% of the fatty acid component of the composition consists of one or more omega-3 fatty acids independently selected from eicosapentaenoic acid (EPA), docosahexaenoic acid (DHA), and docosapentaenoic acid (DPA). In embodiments, the psychiatric disorder is selected from Alzheimer's disease, attention deficit hyperactivity disorder (ADHD) and depression.

In embodiments, the invention provides a method for treating a neuro trauma injury in a subject, the method comprising administering to the subject a therapeutically effective amount of a composition comprising a compound of Formula I, or mixtures thereof, where the amount is effective to treat one or more symptoms of the neuro trauma injury.

In embodiments, at least 50% of the fatty acid component of the composition consists of one or more omega-3 fatty acids independently selected from eicosapentaenoic acid (EPA), docosahexaenoic acid (DHA), and docosapentaenoic acid (DPA).

In one embodiment, the neuro trauma injury is selected from traumatic brain injury, spinal cord injury, ischemic stroke, and concussion.

The invention also provides a method for treating epilepsy or epileptic syndrome by administering to a subject in need of such treatment a composition comprising a compound of Formula IC having gabapentin as the optional non-metal molecule A+. In one embodiment, the method comprises administering to the subject in need of treatment for epilepsy or epileptic syndrome a composition comprising a compound of Formula I and at least one additional API. In one embodiment, the additional API is an anti-epileptic agent such as gabapentin, or a pharmaceutically acceptable salt and prodrug thereof.

### Pain

The disclosure also provides methods for treating or managing pain. In one embodiment, the pain is neuropathic pain and the method comprises administering to a subject in need of treatment for neuropathic pain a pharmaceutical composition comprising an effective amount of a compound of Formula I, or mixtures thereof, and at least one additional API. In one embodiment, the additional API is a non-steroidal anti-inflammatory agent (NSAID), or a pharmaceutically acceptable salt or prodrug thereof.

In one embodiment, the pain is nociceptive pain and the method comprises administering to a subject in need of treatment for nociceptive pain a pharmaceutical composition comprising an effective amount a compound of Formula I, or mixtures thereof, and at least one additional API. In one embodiment, the additional API is gabapentin, or a pharmaceutically acceptable salt or prodrug thereof.

### Combination Therapies

In the context of combination therapies, a composition of the invention may be administered together with at least one additional API or separately from the additional API. Where delivery is together, a composition of the invention may be delivered in the same dosage form as the additional API, or in a different dosage form. One of the advantages of the present invention, as discussed above, is the ease of formulating the compositions described herein with additional APIs and excipients in a single solid dosage form due to their form as a free flowing powder that is chemically and physically stable (as opposed to the relatively unstable oily liquid form of free fatty acids and their esters).

In one embodiment, a composition of the invention is formulated in a single solid dosage form with an antihyperlipidemic agent or an anti-diabetic agent. Antihyperlipidemic agents that may be used include HMG CoA enzyme inhibitors (e.g., statins), cholesterol absorption inhibitors, and cholesterol esterase transfer protein (CETP) inhibitors. In one embodiment, the antihyperlipidemic agent is selected from a statin, a cholesterol absorption inhibitor, a CETP inhibitor, and pharmaceutically-acceptable salts and prodrugs of any of the foregoing. The pharmaceutically acceptable salt may be selected from the group consisting of a propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenyl acetate, phenylpropionate, phenylbutyrate, citrate, lactate, p-hydroxybutyrate, glycolate, tartrate, methanesulfonate, propanesulfonates, naphthalene-1-sulfonate, naphthalene-2-sulfonate, mandelate, hippurate, gluconate, and lactobionate salt.

In one embodiment, the antihyperlipidemic agent is a statin. In one embodiment, the statin is selected from the group consisting of atorvastatin, risuvostatin, simvastatin, pravastatin, and pharmaceutically acceptable salts and prodrugs of any of the foregoing. In one embodiment, the statin is present in an amount ranging from 5 mg to 100 mg. In one embodiment, the statin is pravastatin.

In one embodiment, the antihyperlipidemic agent is a cholesterol absorption inhibitor. In one embodiment, the cholesterol absorption inhibitor is ezetimibe, also known as Zetia.

In one embodiment, the antihyperlipidemic agent is a CETP inhibitor. In one embodiment, the CETP inhibitor is anacetrapib, or a hydrate, or solvate thereof.

In one embodiment, a composition of the invention is formulated in a single solid dosage form with an anti-epileptic agent or an inhibitor of neuropathic pain such as gabapentin, or a pharmaceutically acceptable salt and prodrug thereof.

The invention is further described in the following examples, which do not limit the scope of the invention described in the claims.

### Non-Pharmaceutical Uses

In one embodiment, the invention provides compositions, particularly compositions of Formula I, and mixtures of same, for a non-pharmaceutical use, e.g., for use as a dietary supplement or additive. In accordance with any of the embodiments described herein, the method may comprise administering to the subject an effective amount of a composition comprising a compound of Formula I, wherein the fatty acid component is a polyunsaturated fatty acid. In embodiments, the polyunsaturated fatty acid is selected from a fatty acid of the omega-3, omega-6, omega-7, or omega-9 series. In embodiments, the polyunsaturated fatty acid is an omega-3 fatty acid independently selected from eicosapentaenoic acid (EPA), docosahexaenoic acid (DHA), and docosapentaenoic acid (DPA). In embodiments, at least 50% of the fatty acid component of the composition consists of one or more omega-3 fatty acids independently selected from eicosapentaenoic acid (EPA), docosahexaenoic acid (DHA), and docosapentaenoic acid (DPA). In embodiments, the effective amount is effective to maintain, promote, or improve the general health of the subject.

In one embodiment, the composition may be used in a method to counter a dietary deficiency or nutritional disorder in a subject. In one embodiment, the composition may be used in a method for maintaining, promoting, or improving the general health of a subject

In one embodiment, the method is a method for improving prenatal health. In one embodiment of this method, the composition comprises from 50 mg to 6 g of DHA or total omega-3 fatty acids, and optionally further comprises one or more of a B vitamin, vitamin C, vitamin E, vitamin A, vitamin D, iron, zinc, calcium, iodine, metafolin, methylsulfonylmethane (also known as dimethyl sulfone and methyl sulfone), N-acetyl-L-cysteine, green tea extract (Camellia sinensis), and grape seed extract (Vitis vinifera). In one embodiment, the B vitamin is selected from thiamine (vitamin B-1), riboflavin (vitamin B-2), niacin (vitamin B-3), pantothenic acid (vitamin B-5), biotin (vitamin B-7), and folic acid (vitamin B-9), or any combination of the foregoing

In one embodiment, the method is a method for improving heart health. In one embodiment of this method, the composition comprises from 50 mg to 6 g of EPA or total omega-3 fatty acids, and optionally further comprises one or more of coenzyme Q10, L-carnitine, an antioxidant, a phytoseterol, and a flavonoid.

In one embodiment, the method is a method for improving joint health. In one embodiment of this method, the composition comprises from 50 mg to 6 g of EPA or total omega-3 fatty acids, and optionally further comprises one or more of chondroitin, glucosamine sulfate, calcium, vitamin D3, ginger extract, turmeric, curcumin, collagen, and a non-steroidal anti-inflammatory (NSAID).

In one embodiment, the method is a method for improving eye health. In one embodiment of this method, the composition comprises from 50 mg to 6 g of DHA or total omega-3 fatty acids, and optionally further comprises one or more of vitamin A, vitamin C, vitamin E, calcium, zinc, copper, selenium, a carotenoid, a flavonoid, and folic acid.

In one embodiment, the method is a method for improving cognitive health. In one embodiment of this method, the composition comprises from 50 mg to 6 g of EPA or total omega-3 fatty acids.

The invention is further described and exemplified by the following non-limiting examples.

### EXAMPLES

### Example 1: Preparation of Glycyl-L-Lysine, DHA Salt

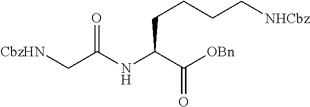 [SMILES: O=C(CNC(=O)OCc1ccccc1)N[C@@H](CCCCNC(=O)OCc1ccccc1)C(=O)OCc1ccccc1]

### Step 1: Benzyl N6-((benzyloxy)carbonyl)-N2-(((benzyloxy)carbonyl)glycyl)-L-lysinate

A solution/suspension of H-Lys(Z)-OBzl hydrochloride (8.0 g, 19.7 mmol) and Z-(L)-Gly-OH (4.11 g, 19.7 mmol) in anhydrous dichloromethane (50 mL) under nitrogen was cooled on ice and treated with HOBT hydrate (3.98 g, 29.5 mmol) and triethylamine (8.25 mL, 59.4 mmol), and the clear solution was stirred for 15 min EDC hydrochloride (4.71 g, 24.6 mmol) was added, and the stirred mixture was allowed to warm to room temperature and stirred 20 h. The product mixture was diluted to 200 mL total volume with dichloromethane, then washed successively with 5% citric acid, water, saturated aqueous sodium bicarbonate, and brine (100 mL each), and dried (Na₂SO₄). The solution was concentrated by half, added directly to a silica gel column and eluted with dichloromethane, then 2:1 dichloromethane/ethyl acetate to afford 10.2 g (93%) of subject material as a clear oil. NMR (CDCl₃): δ 7.37-7.29 (m, 15H) 6.62-6.58 (m, 1H) 5.50-5.40 (m, 1H) 5.20-5.07 (m, 6H) 4.89-4.80 (m, 1H) 4.64-4.61 (m, 1H) 3.87 (d, 2H, J=5 Hz) 3.11-3.08 (m, 2H) 1.90-1.79 (m, 1H) 1.72-1.65 (m, 1H) 1.48-1.40 (m, 2H) 1.30-1.24 (m, 2H)

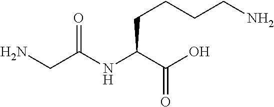 [SMILES: NCCCC[C@H](NC(=O)CN)C(=O)O]

### Step 2: Glycyl-L-Lysine

A stirred solution/suspension of benzyl N6-((benzyloxy)carbonyl)-N2-(((benzyloxy)carbonyl)glycyl)-L-lysinate (10.2 g, 18.2 mmol) in methanol (100 mL) under nitrogen was treated with 20% Pd/C (1.0 g), then evacuated and purged several times with hydrogen via balloon. The mixture was stirred for 24 h under hydrogen, then water (50 mL) was added. The flask was evacuated and purged several times with a hydrogen balloon. The mixture stirred for 48 h more, was evacuated with nitrogen and carefully filtered through Celite with water rinse. The filtrate was concentrated in vacuo to afford a white foam. The foam was dissolved in a minimal amount of water and lyophilized overnight to afford 3.54 g (96%) of (L,L)-Gly-Lys-OH as a white solid. NMR (D₂O): δ 4.21 (dd, 1H, J=5 Hz, 8 Hz) 3.40 (s, 2H) 3.00 (t, 2H, J=7.5 Hz) 1.86-1.66 (m, 4H) 1.46-1.39 (m, 2H)

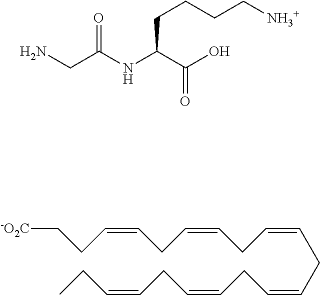

### Step 3: Glycyl-L-Lysine, DHA Salt

A stirred solution/suspension of (L,L)-Gly-Lys-OH (0.32 g, 1.58 mmol) in ethanol (6 mL) was heated to 60° C., then treated with a combined solution of DHA (1.03 g, 3.15 mmol) and alpha-D-tocopherol (17 mg pre-dissolved in 0.3 mL of ethyl acetate and added to the DHA solution) in ethanol (4 mL). The reaction cooled over 20 minutes and was concentrated in vacuo to a foam. The foam was triturated from cold acetonitrile, collected and dried to afford 0.79 g (94%) of (L,L)-Gly-Lys, DHA salt as a white solid. NMR (d₄-MeOH): δ 5.44-5.28 (m, 12H) 4.26 (t, 1H, J=6.5 Hz) 3.57 (d, 2H, J=7 Hz) 2.92-2.80 (m, 12H) 2.39-2.33 (m, 2H) 2.27-2.23 (m, 2H) 2.12-2.04 (m, 2H) 1.88-1.81 (m, 1H) 1.76-1.63 (m, 3H) 1.48-1.42 (m, 2H) 0.96 (t, 3H, J=7.5 Hz)

### Example 2: Preparation of L-Valyl-L-Lysine, DHA Salt

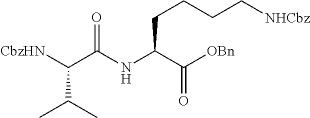 [SMILES: CC(C)[C@H](NC(=O)OCc1ccccc1)C(=O)N[C@@H](CCCCNC(=O)OCc1ccccc1)C(=O)OCc1ccccc1]

### Step 1: Benzyl N6-((benzyloxy)carbonyl)-N2-(((benzyloxy)carbonyl)-L-valyl)-L-lysinate

A solution/suspension of H-Lys(Z)-OBzl hydrochloride (7.0 g, 17.2 mmol) and Z-(L)-Val-OH (4.32 g, 17.2 mmol) in anhydrous dichloromethane (50 mL) under nitrogen was cooled on ice and treated with HOBT hydrate (3.49 g, 25.8 mmol) and triethylamine (7.22 mL, 52.0 mmol), and the clear solution was stirred for 15 min EDC hydrochloride (4.12 g, 21.5 mmol) was added, and the stirred mixture was allowed to warm to room temperature and stirred 20 h. The product mixture was diluted to 200 mL total volume with dichloromethane, then washed successively with 5% citric acid, water, saturated aqueous sodium bicarbonate, and brine (100 mL each), and dried (Na₂SO₄). The solution was concentrated by half, added directly to a silica gel column and eluted with dichloromethane, then 2:1 dichloromethane/ethyl acetate to afford 9.2 g (89%) of subject material as a light yellow solid.

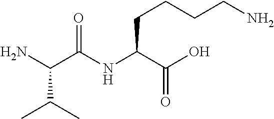 [SMILES: CC(C)[C@H](N)C(=O)N[C@@H](CCCCN)C(=O)O]

### Step 2: L-Valyl-L-Lysine

A stirred solution/suspension of benzyl N6-((benzyloxy)carbonyl)-N2-(((benzyloxy)carbonyl)-L-valyl)-L-lysinate (9.8 g, 16.2 mmol) in methanol (100 mL) under nitrogen was treated with 20% Pd/C (0.98 g), then evacuated and purged several times with hydrogen via balloon. The mixture was stirred for 24 h under hydrogen, then water (50 mL) was added. The flask was evacuated and purged several times with a hydrogen balloon. The mixture stirred for 48 h more, was evacuated with nitrogen and carefully filtered through Celite with water rinse. The filtrate was concentrated in vacuo to afford a yellow foam. The foam was dissolved in a minimal amount of water and lyophilized overnight to afford 3.95 g (99%) of (L,L)-Val-Lys-OH as a yellow solid. NMR (d₄-MeOH): δ 4.25 (dd, 1H, J=5.5 Hz, 7 Hz) 3.26 (d, 1H, J=5 Hz) 2.89 (t, 2H, J=7.5 Hz) 2.10-2.02 (m, 1H) 1.89-1.63 (m, 4H) 1.45-1.41 (m, 2H) 0.99 (d, 3H, J=7 Hz) 0.93 (d, 3H, J=7 Hz)

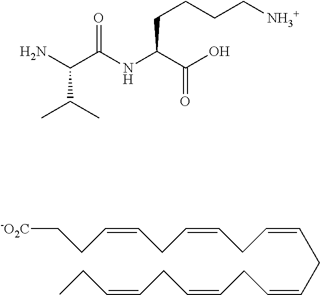

### Step 3: L-Valyl-L-Lysine, DHA Salt

A stirred solution/suspension of (L,L)-Val-Lys-OH (0.30 g, 1.22 mmol) in ethanol (6 mL) was heated to 60° C., then treated with a combined solution of DHA (0.80 g, 2.44 mmol) and alpha-D-tocopherol (14 mg pre-dissolved in 0.3 mL of ethyl acetate and added to the DHA solution) in ethanol (4 mL). The reaction cooled over 20 minutes and was concentrated in vacuo to a foam. The foam was triturated from cold acetonitrile, collected and dried to afford 0.69 g (97%) of (L,L)-Val-Lys, DHA salt as a light yellow solid. NMR (d₄-MeOH): δ 5.44-5.27 (m, 12H) 4.25 (dd, 1H, J=5.5 Hz, 7 Hz) 3.57 (d, 1H, J=5 Hz) 2.93-2.81 (m, 12H) 2.40-2.35 (m, 2H) 2.30-2.26 (m, 2H) 2.21-2.04 (m, 3H) 1.87-1.83 (m, 1H) 1.77-1.60 (m, 3H) 1.48-1.44 (m, 2H) 1.05 (d, 3H, J=7 Hz) 1.01 (d, 3H, J=7 Hz) 0.97 (t, 3H, J=7.5 Hz)

### Example 3: Preparation of L-Leucyl-L-Lysine, DHA Salt

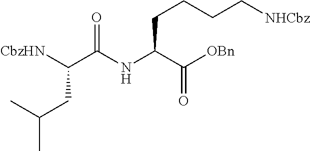 [SMILES: CC(C)C[C@H](NC(=O)OCc1ccccc1)C(=O)N[C@@H](CCCCNC(=O)OCc1ccccc1)C(=O)OCc1ccccc1]

### Step 1: Benzyl N6-((benzyloxy)carbonyl)-N2-(((benzyloxy)carbonyl)-L-leucyl)-L-lysinate

A solution/suspension of H-Lys(Z)-OBzl hydrochloride (7.36 g, 18.1 mmol) and Z-(L)-Leu-OH (4.80 g, 18.1 mmol) in anhydrous dichloromethane (50 mL) under nitrogen was cooled on ice and treated with HOBT hydrate (3.67 g, 27.14 mmol) and triethylamine (7.60 mL, 54.6 mmol), and the clear solution was stirred for 15 min EDC hydrochloride (4.34 g, 22.6 mmol) was added, and the stirred mixture was allowed to warm to room temperature and stirred 20 h. The product mixture was diluted to 200 mL total volume with dichloromethane, then washed successively with 5% citric acid, water, saturated aqueous sodium bicarbonate, and brine (100 mL each), and dried (Na₂SO₄). The solution was concentrated by half, added directly to a silica gel column and eluted with dichloromethane, then 2:1 dichloromethane/ethyl acetate to afford 9.85 g (88%) of subject material as a white solid.

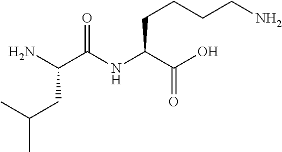 [SMILES: CC(C)C[C@H](N)C(=O)N[C@@H](CCCCN)C(=O)O]

### Step 2: L-Leucyl-L-Lysine

A stirred solution/suspension of benzyl N6-((benzyloxy)carbonyl)-N2-(((benzyloxy)carbonyl)-L-leucyl)-L-lysinate (9.85 g, 15.9 mmol) in methanol (100 mL) under nitrogen was treated with 20% Pd/C (0.98 g), then evacuated and purged several times with hydrogen via balloon. The mixture was stirred for 24 h under hydrogen, then water (50 mL) was added. The flask was evacuated and purged several times with a hydrogen balloon. The mixture stirred for 48 h more, was evacuated with nitrogen and carefully filtered through Celite with water rinse. The filtrate was concentrated in vacuo to afford a white foam. The foam was dissolved in a minimal amount of water and lyophilized overnight to afford 3.78 g (92%) of (L,L)-Leu-Lys-OH as a white solid. NMR (d₄-MeOH): δ 4.24 (dd, 1H, J=5 Hz, 7 Hz) 3.40 (m, 1H) 2.90 (t, 2H, J=7.5 Hz) 1.88-1.83 (m, 1H) 1.75-1.37 (m, 8H) 0.96 (d, 3H, J=6.5 Hz) 0.94 (d, 3H, J=6.5 Hz)

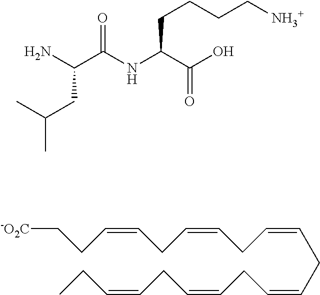

### Step 3: L-Leucyl-L-Lysine, DHA Salt

A stirred solution/suspension of (L,L)-Leu-Lys-OH (0.32 g, 1.24 mmol) in ethanol (6 mL) was heated to 60° C., then treated with a combined solution of DHA (0.81 g, 2.48 mmol) and alpha-D-tocopherol (15 mg pre-dissolved in 0.3 mL of ethyl acetate and added to the DHA solution) in ethanol (4 mL). The reaction cooled over 20 minutes and was concentrated in vacuo to a foam. The foam was triturated from cold acetonitrile, collected and dried to afford 0.68 g (94%) of (L,L)-Leu-Lys, DHA salt as an off-white solid. NMR (d₄-MeOH): δ 5.45-5.26 (m, 12H) 4.22 (dd, 1H, J=5.5 Hz, 7 Hz) 3.72-3.69 (m, 1H) 2.92-2.79 (m, 12H) 2.38-2.33 (m, 2H) 2.27-2.23 (m, 2H) 2.11-2.04 (m, 2H) 1.85-1.82 (m, 1H) 1.77-1.41 (m, 8H) 0.99-0.92 (m, 9H)

### Example 4: Preparation of L-Seryl-L-Lysine, DHA Salt

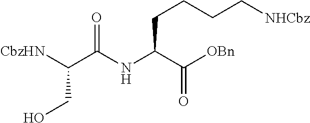 [SMILES: O=C(NCCCC[C@H](NC(=O)[C@H](CO)NC(=O)OCc1ccccc1)C(=O)OCc1ccccc1)OCc1ccccc1]

### Step 1: Benzyl N6-((benzyloxy)carbonyl)-N2-(((benzyloxy)carbonyl)-L-seryl)-L-lysinate

A solution/suspension of H-Lys(Z)-OBzl hydrochloride (7.0 g, 17.2 mmol) and Z-(L)-Ser-OH (4.12 g, 17.2 mmol) in anhydrous dichloromethane (50 mL) under nitrogen was cooled on ice and treated with HOBT hydrate (3.49 g, 25.8 mmol) and triethylamine (7.22 mL, 52.0 mmol), and the clear solution was stirred for 15 min EDC hydrochloride (4.12 g, 21.5 mmol) was added, and the stirred mixture was allowed to warm to room temperature and stirred 20 h. The product mixture was diluted to 200 mL total volume with dichloromethane, then washed successively with 5% citric acid, water, saturated aqueous sodium bicarbonate, and brine (100 mL each), and dried (Na₂SO₄). The solution was concentrated by half, added directly to a silica gel column and eluted with dichloromethane, then 2:1 dichloromethane/ethyl acetate to afford 9.67 g (95%) of subject material as a light yellow solid.

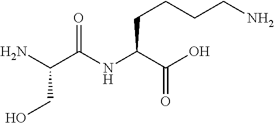 [SMILES: NCCCC[C@H](NC(=O)[C@@H](N)CO)C(=O)O]

### Step 2: L-Seryl-L-Lysine

A stirred solution/suspension of benzyl N6-((benzyloxy)carbonyl)-N2-(((benzyloxy)carbonyl)-L-seryl)-L-lysinate (9.60 g, 16.2 mmol) in methanol (100 mL) under nitrogen was treated with 20% Pd/C (0.96 g), then evacuated and purged several times with hydrogen via balloon. The mixture was stirred for 24 h under hydrogen, then water (50 mL) was added. The flask was evacuated and purged several times with a hydrogen balloon. The mixture stirred for 48 h more, was evacuated with nitrogen and carefully filtered through Celite with water rinse. The filtrate was concentrated in vacuo to afford a yellow foam. The foam was dissolved in a minimal amount of water and lyophilized overnight to afford 3.44 g (91%) of (L,L)-Ser-Lys-OH as a yellow solid. NMR (D₂O): δ 4.23 (dd, 1H, J=5 Hz, 8 Hz) 3.75 (d, 2H, J=5 Hz) 3.56 (t, 1H, J=5 Hz) 3.01 (t, 2H, J=7.5 Hz) 1.89-1.68 (m, 4H) 1.47-1.41 (m, 2H)

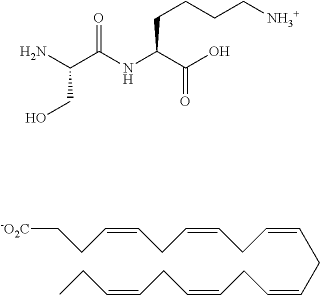

### Step 3: L-Seryl-L-Lysine, DHA Salt

A stirred solution/suspension of (L,L)-Ser-Lys-OH (0.32 g, 1.37 mmol) in ethanol (6 mL) was heated to 60° C., then treated with a combined solution of DHA (0.90 g, 2.74 mmol) and alpha-D-tocopherol (15 mg pre-dissolved in 0.3 mL of ethyl acetate and added to the DHA solution) in ethanol (4 mL). The reaction cooled over 20 minutes and was concentrated in vacuo to a foam. The foam was triturated from cold acetonitrile, collected and dried to afford 0.71 g (92%) of (L,L)-Ser-Lys, DHA salt as a light yellow solid. NMR (d₄-MeOH): δ 5.44-5.26 (m, 12H) 4.25 (dd, 1H, J=5 Hz, 7.5 Hz) 3.80 (dd, 2H, J=1 Hz, 5 Hz) 3.70 (t, 1H, J=5 Hz) 2.92-2.80 (m, 12H) 2.39-2.33 (m, 2H) 2.28-2.24 (m, 2H) 2.11-2.04 (m, 2H) 1.90-1.83 (m, 1H) 1.74-1.64 (m, 3H) 1.48-1.42 (m, 2H) 0.96 (t, 3H, J=7.5 Hz)

### Example 5: Preparation of L-Histidyl-L-Lysine, DHA Salt

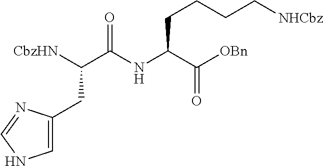 [SMILES: O=C(NCCCC[C@H](NC(=O)[C@H](Cc1c[nH]cn1)NC(=O)OCc1ccccc1)C(=O)OCc1ccccc1)OCc1ccccc1]

### Step 1: Benzyl N6-((benzyloxy)carbonyl)-N2-(((benzyloxy)carbonyl)-L-histidyl)-L-lysinate

A solution/suspension of H-Lys(Z)-OBzl hydrochloride (6.98 g, 17.15 mmol) and Z-(L)-His-OH (4.96 g, 17.15 mmol) in anhydrous dichloromethane (50 mL) under nitrogen was cooled on ice and treated with HOBT hydrate (3.48 g, 25.7 mmol) and triethylamine (7.19 mL, 51.8 mmol), and the clear solution was stirred for 15 min EDC hydrochloride (4.11 g, 21.4 mmol) was added, and the stirred mixture was allowed to warm to room temperature and stirred 20 h. The product mixture was diluted to 200 mL total volume with dichloromethane, then washed successively with 5% citric acid, water, saturated aqueous sodium bicarbonate, and brine (100 mL each), and dried (Na₂SO₄). The solution was concentrated by half, added directly to a silica gel column and eluted with dichloromethane, then 2:1 dichloromethane/ethyl acetate to afford 4.32 g (39%) of subject material as a white solid.

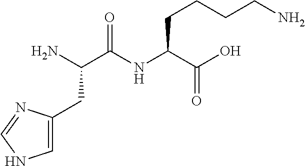 [SMILES: NCCCC[C@H](NC(=O)[C@@H](N)Cc1c[nH]cn1)C(=O)O]

### Step 2: L-Histidyl-L-Lysine

A stirred solution/suspension of benzyl N6-((benzyloxy)carbonyl)-N2-(((benzyloxy)carbonyl)-L-histidyl)-L-lysinate (0.98 g, 1.53 mmol) in methanol (20 mL) under nitrogen was treated with 20% Pd/C (0.09 g), then evacuated and purged several times with hydrogen via balloon. The mixture was stirred for 24 h under hydrogen, then water (10 mL) was added. The flask was evacuated and purged several times with a hydrogen balloon. The mixture stirred for 48 h more, was evacuated with nitrogen and carefully filtered through Celite with water rinse. The filtrate was concentrated in vacuo to afford a white foam. The foam was dissolved in a minimal amount of water and lyophilized overnight to afford 0.4 g (93%) of (L,L)-His-Lys-OH as a white solid. NMR (d₄-MeOH): δ 7.58 (d, 1H, J=1 Hz) 6.84 (s, 1H) 4.23-4.18 (m, 1H) 3.61-3.58 (m, 1H) 3.00-2.79 (m, 4H) 1.89-1.63 (m, 4H) 1.45-1.41 (m, 2H)

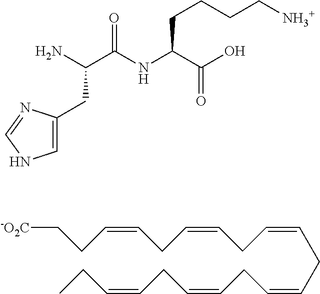

### Step 3: L-Histidyl-L-Lysine, DHA Salt

A stirred solution/suspension of (L,L)-His-Lys-OH (0.13 g, 0.46 mmol) in ethanol (4 mL) was heated to 60° C., then treated with a combined solution of DHA (0.30 g, 0.92 mmol) and alpha-D-tocopherol (6 mg pre-dissolved in 0.3 mL of ethyl acetate and added to the DHA solution)

in ethanol (2 mL). The reaction cooled over 20 minutes and was concentrated in vacuo to a foam. The foam was triturated from cold acetonitrile, collected and dried to afford 0.23 g (82%) of (L,L)-His-Lys, DHA salt as an off white solid. NMR (d₄-MeOH): δ 7.68 (d, 1H, J=6 Hz) 6.93 (s, 1H) 5.43-5.27 (m, 12H) 4.23-4.19 (m, 1H) 3.81 (t, 1H, J=6 Hz) 3.08-3.03 (m, 2H) 2.92-2.80 (m, 12H) 2.40-2.33 (m, 2H) 2.26-2.23 (m, 2H) 2.11-2.04 (m, 2H) 1.87-1.84 (m, 1H) 1.73-1.61 (m, 3H) 1.43-1.38 (m, 2H) 0.96 (t, 3H, J=7.5 Hz)

### Example 6: Preparation of L-Leucine Metformin Eicosapentaenoic Acid (EPA) Salt

### Step 1: Preparation of Metformin Eicosapentaenoic Acid (EPA) Salt

A solution of metformin free base (2.76 g, 21.4 mmol) in methanol (40 mL) was heated to 55° C., then a solution of EPA (6.05 g, 20 mmol) in methanol (25 mL) which had been treated with alpha-D-tocopherol (120 mg in 0.4 mL of ethyl acetate) was added in one portion. The mixture was stirred a few minutes more and cooled to room temperature over 20 min and concentrated in vacuo. The residue was triturated with acetonitrile (50 mL), collected and dried in vacuo at room temperature to afford 7.75 g (90%) of Metformin EPA salt as a waxy white solid. ¹H NMR (400 MHz, d4 MeOH): δ 5.40-5.29 (m, 10H) 3.02 (s, 6H) 2.85-2.79 (m, 8H) 2.18-2.05 (m, 6H) 1.67-1.63 (m, 2H) 0.96 (t, 3H, J=7.5 Hz).

### Step 2:

A stirred solution of metformin EPA salt (4.32 g, 10 mmol) in ethanol (150 mL) under nitrogen was heated to 70° C., then treated with a hot solution of L-leucine (1.31 g, 10 mmol) in water (50 mL, 40 mL to dissolve, 10 mL to rinse). The mixture was stirred 30 min, cooled and concentrated in vacuo. The residue was triturated from cold acetonitrile, collected, and dried in vacuo to afford 5.12 g (91%) of L-leucine metformin EPA salt as a white solid. ¹H NMR (400 MHz, d4 AcOH): δ 5.43-5.30 (m, 10H) 4.05-4.01 (m, 1H) 3.11 (s, 6H) 2.88-2.82 (m, 8H) 2.38 (t, 2H, J=7.5 Hz) 2.17-2.06 (m, 4H) 1.86-1.68 (m, 5H) 0.98-0.95 (m, 9H).

### Example 7: Preparation of L-Leucine Metformin Docosahexaenoic Acid (DHA) Salt

### Step 1: Preparation of Metformin Docosahexaenoic Acid (DHA) Salt

A solution of metformin free base (2.76 g, 21.4 mmol) in methanol (40 mL) was heated to 55° C., then a solution of DHA (6.57 g, 20 mmol) in methanol (25 mL) which had been treated with alpha-D-tocopherol (120 mg in 0.4 mL of ethyl acetate) was added in one portion. The mixture was stirred a few minutes more and cooled to room temperature over 20 min and concentrated in vacuo. The residue was triturated with acetonitrile (50 mL), collected and dried in vacuo at room temperature to afford 8.09 g (88%) of Metformin DHA salt as a waxy white solid. ¹H NMR (400 MHz, d4 MeOH): δ 5.33-5.27 (m, 12H) 3.02 (s, 6H) 2.87-2.79 (m, 10H) 2.38-2.33 (m, 2H) 2.21-2.17 (m, 2H) 2.11-2.03 (m, 2H) 0.96 (t, 3H, J=7.5 Hz).

### Step 2:

A stirred solution of metformin DHA salt (458 mg, 1 mmol) in ethanol (16 mL) under nitrogen was heated to 70° C., then treated with a hot solution of L-leucine (131 mg, 1 mmol) in water (5 mL). 3 mL of water was used to introduce the solution, and the remaining 2 mL used to rinse the glassware used to dissolve the leucine. The mixture was stirred for 30 min, cooled to room temperature and concentrated in vacuo. The residue was triturated from cold acetonitrile, collected and dried in vacuo to afford 524 mg (89%) of L-Leucine metformin DHA salt as a white solid. 1H NMR (400 MHz, d4 AcOH): δ 5.42-5.30 (m, 12H) 4.04-4.00 (m, 1H) 3.11 (s, 6H) 2.92-2.82 (m, 10H) 2.44-2.40 (m, 4H) 2.12-2.04 (m, 2H) 1.86-1.72 (m, 3H) 0.98-0.95 (m, 9H).

### Example 8: Preparation of L-Leucine Metformin Docosapentaenoic Acid (DPA) Salt

### Step 1: Preparation of Metformin Docosapentaenoic Acid (DPA) Salt

A solution of metformin free base (1.18 g, 9.14 mmol) in methanol (15 mL) was heated to 55° C., then a solution of DPA (2.81 g, 8.5 mmol) in methanol (10 mL) which had been treated with alpha-D-tocopherol (50 mg in 0.2 mL of EtOAc) was added in one portion. The mixture was stirred a few minutes more and cooled to room temperature over 20 min and concentrated in vacuo. The residue was triturated with acetonitrile (25 mL), collected and dried in vacuo at room temperature to afford 3.40 g (87%) of metformin DPA salt as a waxy white solid. 1H NMR (400 MHz, d4 MeOH): δ 5.39-5.28 (m, 10H) 3.02 (s, 6H) 2.86-2.80 (m, 8H) 2.16-2.04 (m, 6H) 1.62-1.58 (m, 2H) 1.40-1.33 (m, 4H) 0.96 (t, 3H, J=7.5 Hz).

### Step 2:

A stirred solution of metformin DPA salt (460 mg, 1 mmol) in ethanol (15 mL) under nitrogen was heated to 70° C., then treated with a hot solution of L-leucine (131 mg, 1 mmol) in water (5 mL). 3 mL of water was used to introduce the solution, and the remaining 2 mL used to rinse the glassware used to dissolve the leucine. The mixture was stirred 30 min, cooled to room temperature and concentrated in vacuo. The residue was triturated from cold acetonitrile, collected and dried in vacuo to afford 514 mg (87%) of L-Leucine metformin DPA salt as a white solid. ¹H NMR (400 MHz, d4 AcOH): δ 5.43-5.31 (m, 10H) 4.04-4.00 (m, 1H) 3.12 (s, 6H) 2.88-2.82 (m, 8H) 2.36 (t, 2H, J=7.5 Hz) 2.13-2.07 (m, 4H) 1.85-1.62 (m, 5H) 1.42-1.36 (m, 4H) 0.99-0.95 (m, 9H).

### EQUIVALENTS

Those skilled in the art will recognize or be able to ascertain using no more than routine experimentation, many equivalents to the specific embodiments of the invention described herein. Such equivalents are intended to be encompassed by the following claims.

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

1. A compound of Formula I, or an enantiomer, polymorph, solvate, or hydrate thereof: 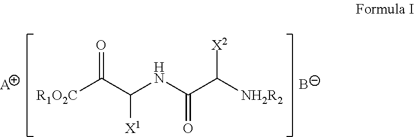
wherein
R₁ is H when A is absent, or absent when A is present,
R₂ is H or absent,
X₁ and X₂ may be the same or different and are each the side chain of an amino acid residue,
A+ is present or absent and is a monovalent metal cation, or a non-metal molecule having at least one basic function, and
B− is an unsaturated fatty acid molecule.

2. The compound of claim 1, wherein A⁺ is absent and R₁ is H.

3. The compound of claim 1, wherein R₁ is absent, and A+ is a monovalent metal cation, a monovalent amine-based cation, metformin, or gabapentin.

4. The compound of claim 3, wherein the monovalent metal cation is selected from Na⁺ and K⁺.

5. (canceled)

6. The compound of claim 1, wherein the fatty acid is a medium chain (C₈-C₁₂) or a long chain (C₁₆-C₂₄) polyunsaturated fatty acid.

7. (canceled)

8. (canceled)

9. (canceled)

10. The compound of claim 6, wherein the long chain polyunsaturated fatty acid is a (C₁₆-C₂₄) fatty acid of the omega-3 series selected from hexadecatrienoic acid (HTA), alpha-linolenic acid (ALA), stearidonic acid (SDA), eicosatrienoic acid (ETE), eicosatetraenoic acid (ETA), eicosapentaenoic acid (EPA, timnodonic acid), heneicosapentaenoic acid (HPA), docosapentaenoic acid (DPA, clupanodonic acid), docosahexaenoic acid (DHA, Cervonic acid), tetracosapentaenoic acid, 24:5 (n-3), and tetracosahexaenoic acid (Nisinic acid), 24:6 (n-3).

11. The compound of claim 10, wherein the omega-3 fatty acid is selected from eicosapentaenoic acid (EPA), docosahexaenoic acid (DHA), and docosapentaenoic acid (DPA).

12. (canceled)

13. The compound of claim 6, wherein the long chain polyunsaturated fatty acid is a (C₁₆-C₂₄) fatty acid of the omega-6 series selected from linoleic acid (LA), gamma-linolenic acid (GLA), eicosadienoic acid, dihomo-gamma-linolenic acid (DGLA), arachidonic acid (AA), docosadienoic acid, adrenic acid, docosapentaenoic acid (Osbond acid), tetracosatetraenoic acid, and tetracosapentaenoic acid, 24:5 (n-6).

14. (canceled)

15. The compound of claim 6, wherein the long chain polyunsaturated fatty acid is a (C₁₆-C₂₄) fatty acid of the omega-9 series selected from mead acid, 20:3 (n-9), all-cis-5,8,11-eicosatrienoic acid. In one embodiment, the monounsaturated omega-9 fatty acid is selected from the group consisting of oleic acid, eicosenoic acid, erucic acid, and nervonic acid.

16. A composition comprising the compound of claim 1.

17. The composition of claim 16, wherein the composition is a pharmaceutical composition, optionally comprising a pharmaceutically acceptable carrier or excipient.

18. A unit dosage form comprising the compound of claim 1, wherein the unit dosage form comprises from about 0.05 g to 12 g of total fatty acids in the fatty acid component of the compound or mixture.

19. The pharmaceutical composition of claim 17, wherein the composition further comprises one or more additional active pharmaceutical agents (APIs).

20. The pharmaceutical composition of claim 19, wherein the one or more additional APIs is selected from the group consisting of an antihyperlipidemic agent, an anti-diabetic agent, an anti-epileptic agent, and an anti-inflammatory agent, and combinations thereof.

21. The pharmaceutical composition of claim 20, wherein the antihyperlipidemic agent is selected from an HMG CoA enzyme inhibitor, a cholesterol absorption inhibitor, and a cholesterol esterase transfer protein (CETP) inhibitor, and combinations thereof.

22. The pharmaceutical composition of claim 20, wherein the antihyperlipidemic agent is a statin.

23. The pharmaceutical composition of claim 22, wherein the statin is selected from the group consisting of atorvastatin, risuvostatin, simvastatin, pravastatin, and pharmaceutically acceptable salts or prodrugs thereof.

24. The pharmaceutical composition of claim 20, wherein the anti-inflammatory agent is an NSAID.

25. (canceled)

26. (canceled)

27. A method for treating a disease or disorder responsive to treatment with a polyunsaturated fatty acid, the method comprising administering to a subject in need thereof the compound of claim 1, wherein the disease or disorder is selected from a gastrointestinal inflammatory disease or disorder, a metabolic disease or disorder, a cardiovascular disease or disorder, a hematological disorder, cancer, an inflammatory disease or disorder, and a neurological disease or disorder.

28. The method of claim 27, wherein the metabolic disease or disorder is abnormal glucose metabolism manifesting in diabetes, including type 2 diabetes, or pre-diabetes, insulin resistance, abnormal lipid metabolism manifesting as hypertriglyceridemia, i.e., elevated triglycerides, mixed dyslipidemia, hypercholesterolemia, fatty liver, and combined abnormal glucose and lipid metabolism manifesting in obesity; or a dyslipidemic disorder selected from hypertriglyceridemia, hypercholesterolemia and mixed dyslipidemias.

29. (canceled)

30. (canceled)

31. (canceled)

32. The method of claim 27, wherein the inflammatory disease or disorder is arthritis, inflammatory bowel disease, or psoriasis.

33. (canceled)

34. (canceled)

35. (canceled)

36. The method of claim 27, wherein the gastrointestinal inflammatory disease or disorder is postoperative intestinal inflammation, postoperative ileus, ischemia reperfusion injury, or a combination thereof.

37. (canceled)
